(19) United States
(12) Patent Application Publication
(10) Pub. No.: US 2010/0311790 A1
(43) Pub. Date: Dec. 9, 2010

(54) AMIDE COMPOUNDS AND PLANT DISEASE CONTROLLING METHOD USING SAME

(75) Inventors: Hiroshi Sakaguchi, Toyonaka-shi (JP); Mayumi Kubota, Toyonaka-shi (JP)

Correspondence Address: SUGHRUE MION, PLLC, 2100 PENNSYLVANIA AVENUE, N.W., SUITE 800, WASHINGTON, DC, 20037, US

(73) Assignee: SUMITOMO CHIEMCAL COMPANY LIMITED, CHUO-KU TOKYO (JP)

(21) Appl. No.: 12/744,767

(22) PCT Filed: Nov. 21, 2008
(86) PCT No.: PCT/JP2008/071743

§ 371 Date: May 26, 2010

(30) Foreign Application Priority Data
Nov. 29, 2007 (JP) 2007-308496
Nov. 29, 2007 (JP) 2007-308497
Nov. 29, 2007 (JP) 2007-3084999
Nov. 29, 2007 (JP) 2007308498

(51) Int. Cl. A01N 43/42; C07D 215/48; A01P 15/00; C07C 217/58

(52) U.S. Cl. 514/311; 546/169; 564/389

(57) ABSTRACT

The object of the present invention is to provide a compound having excellent controlling effect on plant diseases. An amide compound represented by the following formula (I) has excellent controlling effect on plant diseases:

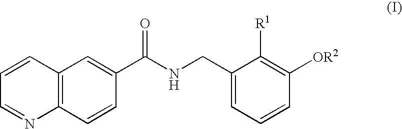

wherein R¹ is a hydrogen atom or a fluorine atom, and R² is a C3-C8 linear alkenyl group or a C3-C8 linear alkynyl group.

### TECHNICAL FIELD

The present invention relates to amide compounds and a method for controlling plant diseases using the same.

### BACKGROUND ART

Hitherto, many compounds have been developed and put to practical use as active ingredients of plant disease controlling agents. However, these compounds do not always have sufficient controlling effect.

[Patent Document 1] WO 2005/033079

### DISCLOSURE OF THE INVENTION

### Problem to be Solved by the Invention

The object of the present invention is to provide a compound having excellent controlling effect for plant diseases.

### Means for Solving the Problem

The present inventors investigated in order to find a compound having an excellent controlling effect on plant diseases, and consequently found that an amide compound represented by the formula (I) shown below has an excellent controlling effect on plant diseases, whereby the present invention has been accomplished.

The present invention provides an amide compound represented by the following formula (I):

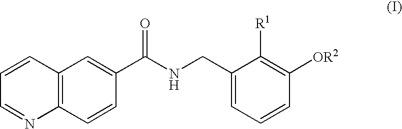

wherein R¹ is a hydrogen atom or a fluorine atom, and R² is a C3-C8 linear alkenyl group or a C3-C8 linear alkynyl group (the amide compound is hereinafter referred to as “invented compound”), a composition for controlling plant diseases which comprises the invented compound as an active ingredient (the composition is hereinafter referred to as “invented controlling agent”), and a method for controlling plant diseases which comprises a step of applying an effective amount of the invented compound to plants or soil (the method is hereinafter referred to as “invented controlling method”).

The present invention also provides an amine compound represented by the formula (III), or its salts:

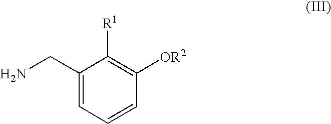

wherein R¹ is a hydrogen atom or a fluorine atom, and R² is a C3-C8 linear alkenyl group or a C3-C8 linear alkynyl group. This compound represented by the formula (III) is hereinafter referred to as the present amine compound in some cases.

### ADVANTAGES OF THE INVENTION

The invented compound has an excellent controlling effect for plant diseases, and hence it is useful as an active ingredient of compositions for controlling plant diseases.

### BEST MODE FOR CARRYING OUT THE INVENTION

The C3-C8 linear alkenyl group represented by R² includes propenyl groups, linear butenyl groups, linear pentenyl groups, linear hexenyl groups, linear heptenyl groups and linear octenyl groups. Specific examples of the propenyl groups include 2-propenyl group. Specific examples of the linear butenyl groups include 2-butenyl group, and 3-butenyl group. Specific examples of the linear pentenyl groups include 2-pentenyl group, 3-pentenyl group, and 4-pentenyl group. Specific examples of the linear hexenyl groups include 2-hexenyl group, 3-hexenyl group, and 4-hexenyl group, 5-hexenyl group. Specific examples of the linear heptenyl groups include 2-heptenyl group, 3-heptenyl group, 4-heptenyl group, 5-heptenyl group, and 6-heptenyl group. Specific examples of the linear octenyl groups include 2-octenyl group, 3-octenyl group, 4-octenyl group, 5-octenyl group, 6-octenyl group, and 7-octenyl group.

The C3-C8 linear alkynyl group represented by R² includes propynyl groups, linear butynyl groups, linear pentynyl groups, linear hexynyl groups, linear heptynyl groups and linear octynyl groups. Specific examples of the propynyl groups include 2-propynyl group. Specific examples of the linear butynyl groups include 2-butynyl group, and 3-butynyl group. Specific examples of the linear pentynyl groups include 2-pentynyl group, 3-pentynyl group, and 4-pentynyl group. Specific examples of the linear hexynyl groups include 2-hexynyl group, 3-hexynyl group, 4-hexynyl group, and 5-hexynyl group. Specific examples of the linear heptynyl groups include 2-heptynyl group, 3-heptynyl group, 4-heptynyl group, 5-heptynyl group, and 6-heptynyl group. Specific examples of the linear octynyl groups include 2-octynyl group, 3-octynyl group, 4-octynyl group, 5-octynyl group, 6-octynyl group, and 7-octynyl group.

Preferable examples of the C3-C8 linear alkenyl group represented by R² are the linear pentenyl groups, linear hexenyl groups and linear heptenyl groups. More preferable examples are 4-pentenyl group, 5-hexenyl group and 6-heptenyl group.

Preferable examples of the C3-C8 linear alkynyl group represented by R² are the linear pentynyl groups, linear hexynyl groups and linear heptynyl groups. More preferable examples thereof are 4-pentynyl group, 5-hexynyl group and 6-heptynyl group.

Processes for producing the invented compound are explained below.

The invented compound can be produced, for example, by any of the following (Process 1) to (Process 3).

### (Process 1)

The invented compound can be produced by causing a compound (II) to react with a compound (III) or its salt (e.g. hydrochloride and hydrobromide) in the presence of a dehydrating-condensation agent:

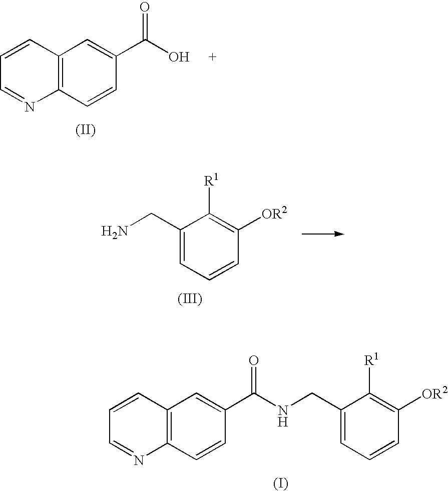

wherein R¹ is a hydrogen atom or a fluorine atom, and R² is a C3-C8 linear alkenyl group or a C3-C8 linear alkynyl group.

This reaction is usually carried out in the presence of a solvent.

The solvent used in the reaction includes ethers such as tetrahydrofuran (hereinafter referred to as THF in some cases), ethylene glycol dimethyl ether, and tert-butyl methyl ether (hereinafter referred to as MTBE in some cases); aliphatic hydrocarbons such as hexane, heptane, and octane; aromatic hydrocarbons such as toluene, and xylene; halogenated hydrocarbons such as chlorobenzene; esters such as butyl acetate, and ethyl acetate; nitriles such as acetonitrile; acid amides such as N,N-dimethylformamide (hereinafter referred to as DMF in some cases); sulfoxides such as dimethyl sulfoxide (hereinafter referred to as DMSO in some cases); nitrogen-containing aromatic compounds such as pyridine; and mixtures thereof.

The dehydrating-condensation agent used in the reaction includes carbodiimides such as 1-ethyl-3-(3-dimethylaminopropyl)carbodiimide hydrochloride (hereinafter referred to as WSC), and 1,3-dicyclohexylcarbodiimide; and (benzotriazol-1-yloxy)tris(dimethylamino)phosphonium hexafluorophosphate (hereinafter referred to as BOP reagent in some cases).

The compound (III) is usually used in an amount of 0.5 to 3 moles per mole of the compound (II), and the dehydrating-condensation agent is usually used in an amount of 1 to 5 moles per mole of the compound (II).

The reaction temperature ranges usually from −20° C. to 140° C., and the reaction time ranges usually from 1 to 24 hours.

After completion of the reaction, the invented compound can be isolated by adding water to the reaction mixture and treating the resulting mixture as follows. When a solid precipitates, the mixture is filtered. When no solid precipitates, the mixture is extracted with an organic solvent and the organic layer is subjected to post-treatments such as drying and concentration. The invented compound isolated may be further purified by operations such as chromatography, and recrystallization.

### (Process 2)

The invented compound can be produced by causing a compound (IV) or its salt (e.g. hydrochloride) to react with a compound (III) or its salt (e.g. hydrochloride and hydrobromide) in the presence of a base:

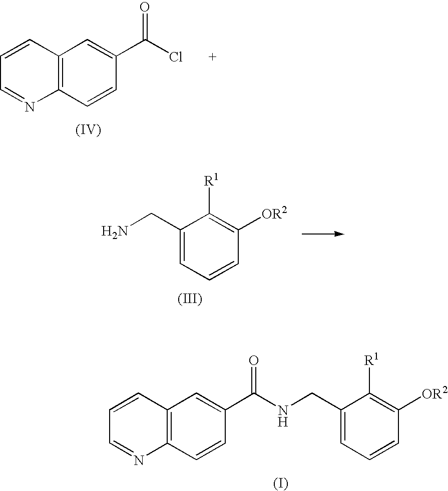

wherein R¹ and R² are as defined above.

This reaction is usually carried out in the presence of a solvent.

The solvent used in the reaction includes ethers such as THF, ethylene glycol dimethyl ether, and MTBE; aliphatic hydrocarbons such as hexane, heptane, and octane; aromatic hydrocarbons such as toluene, and xylene; halogenated hydrocarbons such as chlorobenzene; esters such as butyl acetate, and ethyl acetate; nitriles such as acetonitrile; and mixtures thereof.

The base used in the reaction includes alkali metal carbonates such as sodium carbonate, and potassium carbonate; tertiary amines such as triethylamine, and diisopropylethylamine; and nitrogen-containing aromatic compounds such as pyridine, and 4-dimethylaminopyridine.

The compound (III) is usually used in an amount of 0.5 to 3 moles per mole of the compound (IV), and the base is usually used in an amount of 1 to 5 moles per mole of the compound (IV).

The reaction temperature ranges usually from −20° C. to 100° C., and the reaction time ranges usually from 0.1 to 24 hours.

After completion of the reaction, the invented compound can be isolated by adding water to the reaction mixture and treating the resulting mixture as follows. When a solid precipitates, the mixture is filtered. When no solid precipitates, the mixture is extracted with an organic solvent and the organic layer is subjected to post-treatments such as drying and concentration. The invented compound isolated may be further purified by operations such as chromatography, and recrystallization.

### (Process 3)

The invented compound can be produced by causing a compound (V) to react with a compound (VI) in the presence of a base:

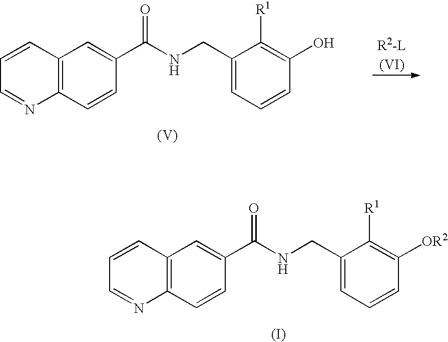

wherein R² and R² are as defined above, and L is a chlorine atom, a bromine atom, an iodine atom, a methanesulfonyloxy group, a trifluoromethanesulfonyloxy group or a p-toluenesulfonyloxy group.

This reaction is usually carried out in the presence of a solvent.

The solvent used in the reaction includes ethers such as THF, ethylene glycol dimethyl ether, and MTBE; aromatic hydrocarbons such as toluene, and xylene; halogenated hydrocarbons such as chlorobenzene; nitriles such as acetonitrile; acid amides such as DMF; sulfoxides such as dimethyl sulfoxide; ketones such as acetone, methyl ethyl ketone, and methyl isobutyl ketone; water; and mixtures thereof.

The base used in the reaction includes alkali metal carbonates such as sodium carbonate, potassium carbonate, and cesium carbonate; alkali metal hydroxides such as sodium hydroxide; and alkali metal hydrides such as sodium hydride.

The compound (VI) is usually used in an amount of 1 to 10 moles per mole of the compound (V), and the base is usually used in an amount of 1 to 5 moles per mole of the compound (V).

The reaction temperature ranges usually from −20° C. to 100° C., and the reaction time ranges usually from 0.1 to 24 hours.

After completion of the reaction, the invented compound can be isolated by adding water to the reaction mixture and treating the resulting mixture as follows. When a solid precipitates, the mixture is filtered. When no solid precipitates, the mixture is extracted with an organic solvent and the organic layer is subjected to post-treatments such as drying and concentration. The invented compound isolated can be further purified by operations such as chromatography, recrystallization, etc.

A process for producing the present amine compound represented by the formula (III) is explained below.

The present amine compound (III) can be synthesized, for example, by the following (synthesis process).

### (Synthesis Process)

The present amine compound (III) can be produced by removing the protecting group Z of a compound (VIII).

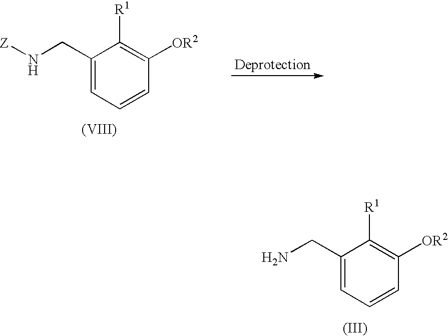

wherein R¹ is a hydrogen atom or a fluorine atom; R² is a C3-C8 linear alkenyl group or a C3-C8 linear alkynyl group; and Z is a protecting group such as 1,1-dimethylethyl carbamate group, and 1,1-dimethyl-2-phenylethyl carbamate group.

For example, when Z is a 1,1-dimethylethyl carbamate group and is removed with an acid, the reaction is usually carried out in the presence of a solvent. The solvent used in the reaction includes aromatic hydrocarbons such as toluene, and xylene; halogenated hydrocarbons such as methylene chloride, chloroform, and chlorobenzene; sulfoxides such as dimethyl sulfoxide; alcohols such as methanol, ethanol, and 2-methylethanol; ketones such as acetone, methyl ethyl ketone, and methyl isobutyl ketone; water; and mixtures thereof.

The acid used in the reaction includes inorganic acids such as hydrochloric acid, and sulfuric acid; and organic acids such as trifluoroacetic acid, p-toluenesulfonic acid, and methanesulfonic acid.

The acid is usually used in an amount of 1 to 10 moles per mole of the compound (VIII).

The reaction temperature ranges usually from 0 to 150° C., and the reaction time ranges usually from 0.1 to 24 hours.

After completion of the reaction, the present amine compound can be obtained in the form of a salt by concentrating the reaction mixture. It is also possible to isolate the present amine compound by extracting the present amine compound with an organic solvent and subjecting the organic layer to post-treatments such as drying and concentration.

Processes for producing each of the intermediates for production in the present invention are explained below.

The compound (V) can be produced by causing a compound (II) to react with a compound (VII) or its salt (e.g. hydrochloride and hydrobromide) in the presence of a dehydrating-condensation agent:

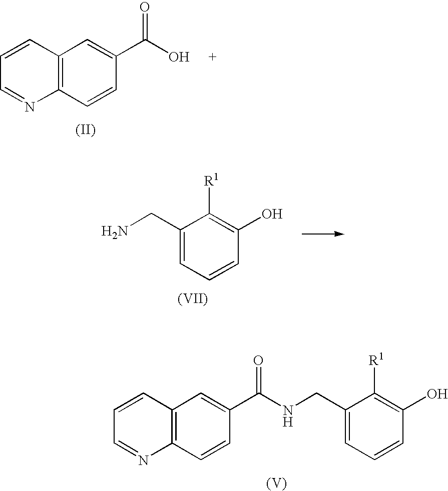

wherein R¹ is as defined above.

This reaction is usually carried out in the presence of a solvent.

The solvent used in the reaction includes ethers such as THF, ethylene glycol dimethyl ether, and MTBE; aliphatic hydrocarbons such as hexane, heptane, and octane; aromatic hydrocarbons such as toluene, and xylene; halogenated hydrocarbons such as chlorobenzene; esters such as butyl acetate, and ethyl acetate; nitriles such as acetonitrile; acid amides such as DMF; sulfoxides such as DMSO; nitrogen-containing aromatic compounds such as pyridine; and mixtures thereof.

The dehydrating-condensation agent used in the reaction includes carbodiimides such as WSC, and 1,3-dicyclohexylcarbodiimide; and BOP reagent.

The compound (VII) is usually used in an amount of 0.5 to 3 moles per mole of the compound (II), and the dehydrating-condensation agent is usually used in an amount of 1 to 5 moles per mole of the compound (II).

The reaction temperature ranges usually from −20° C. to 140° C., and the reaction time ranges usually from 1 to 24 hours.

After completion of the reaction, the compound (V) can be isolated by adding water to the reaction mixture and treating the resulting mixture as follows. When a solid precipitates, the mixture is filtered. When no solid precipitates, the mixture is extracted with an organic solvent and the organic layer is subjected to post-treatments such as drying and concentration. The compound (V) isolated can be further purified by operations such as chromatography, and recrystallization.

The compound (V) can be produced also by causing a compound (IV) or its salt (e.g. hydrochloride) to react with a compound (VII) or its salt (e.g. hydrochloride and hydrobromide) in the presence of a base:

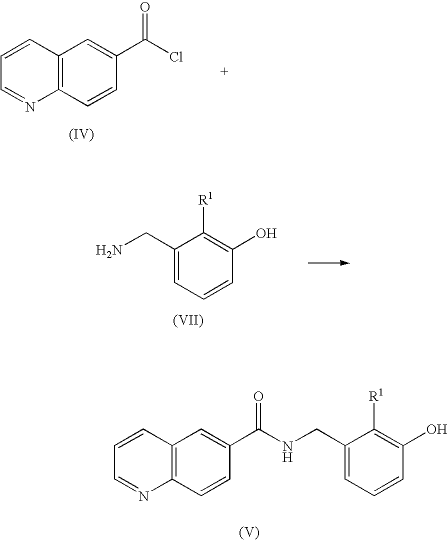

wherein R¹ is as defined above.

This reaction is usually carried out in the presence of a solvent.

The solvent used in the reaction includes ethers such as THF, ethylene glycol dimethyl ether, and MTBE; aliphatic hydrocarbons such as hexane, heptane, and octane; aromatic hydrocarbons such as toluene, and xylene; halogenated hydrocarbons such as chlorobenzene; esters such as butyl acetate, and ethyl acetate; nitriles such as acetonitrile; and mixtures thereof.

The base used in the reaction includes alkali metal carbonates such as sodium carbonate, and potassium carbonate; tertiary amines such as triethylamine, and diisopropylethylamine; and nitrogen-containing aromatic compounds such as pyridine, and 4-dimethylaminopyridine.

The compound (VII) is usually used in an amount of 0.5 to 1 mole per mole of the compound (IV), and the base is usually used in an amount of 1 to 5 moles per mole of the compound (IV).

The reaction temperature ranges usually from −20° C. to 100° C., and the reaction time ranges usually from 0.1 to 24 hours.

After completion of the reaction, the compound (V) can be isolated by adding water to the reaction mixture and treating the resulting mixture as follows. When a solid precipitates, the mixture is filtered. When no solid precipitates, the mixture is extracted with an organic solvent and the organic layer is subjected to post-treatments such as drying and concentration. The compound (V) isolated may be further purified by operations such as chromatography, and recrystallization.

The compound (II), the compound (IV) and its salt, which are used for producing the invented compound, are commercially available or are disclosed in literature.

The compound (VIII) can be produced from, for example, a compound (IX) according to the following scheme:

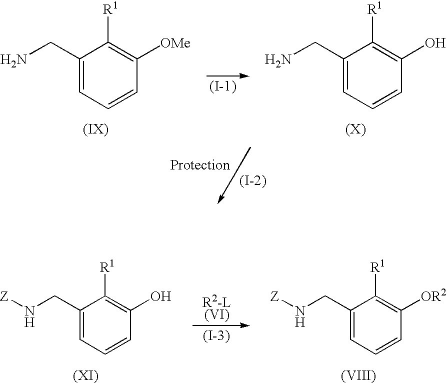

wherein R¹, R², L and Z are as defined above.

### Step (I-1)

A compound (X) can be synthesized by demethylating the methoxy group of the compound (IX).

For example, when the methoxy group is demethylated with an acid, the reaction can be carried out in the presence of a solvent including water and organic solvents such as alcohol solvents (e.g. methanol, ethanol and isopropyl alcohol), acetic acid and trifluoroacetic acid, while it can be carried out without a solvent.

The acid used in the reaction includes inorganic acids such as hydrochloric acid, hydrobromic acid, and sulfuric acid.

The acid is usually used in an amount of 2 to 20 moles per mole of the compound (IX).

The reaction temperature ranges usually from 20 to 150° C., and the reaction time ranges usually from 0.1 to 24 hours.

After completion of the reaction, the compound (X) can be obtained in the form of a salt by concentrating the reaction mixture.

Among compounds (IX), a compound in which R¹ is a hydrogen atom is commercially available. As to a compound (IX) in which R¹ is a fluorine atom, namely a compound (IX-2), U.S. Pat. No. 4,594,092 describes the employment of the compound as a starting material, but the patent fails to specifically describe a production process and physical property values of the compound. The compound (IX-2) can be produced through the following route, which is disclosed in Journal of Organic Chemistry, Vol. 53, No. 5, pp. 1064-1071, 1988.

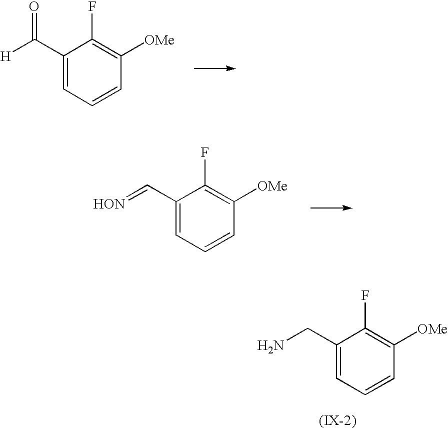

### Step (I-2)

The compound (XI) can be synthesized by protecting the amino group of the compound (X) to form a carbamic acid ester.

For example, when Z is a 1,1-dimethylethyl carbamate group, the reaction is usually carried out in the presence of a solvent. The solvent used in the reaction includes ether solvents such as THF, MTBE, and dioxane; aromatic hydrocarbons such as toluene, and xylene; saturated hydrocarbons such as hexane, heptane, and octane; halogenated hydrocarbons such as methylene chloride, chloroform, and chlorobenzene; sulfoxides such as dimethyl sulfoxide; ketones such as acetone, methyl ethyl ketone, and methyl isobutyl ketone; basic solvents such as pyridine; and mixtures thereof.

The base used in the reaction includes carbonates such as sodium carbonate, and potassium carbonate; tertiary amines such as triethylamine, diisopropylethylamine, 1,8-diazabicyclo[5,4,0]undec-7-ene, and 1,5-diazabicyclo[4,3,0]non-5-ene; and nitrogen-containing aromatic compounds such as pyridine, and 4-dimethylaminopyridine. It is also possible to use dimethylaminopyridine as a catalyst.

Di-tert-butyl dicarbonate is usually used in an amount of 1 to 2 moles per mole of the compound (X).

The reaction temperature ranges usually from −20° C. to 150° C., and the reaction time ranges usually from 0.1 to 24 hours.

After completion of the reaction, the compound (XI) can be isolated by adding water to the reaction mixture, extracting the compound (XI) with an organic solvent and subjecting the organic layer to post-treatments such as drying and concentration. The compound (XI) isolated may be further purified by operations such as chromatography, and recrystallization.

### Step (I-3)

The compound (VIII) can be synthesized by reacting the compound (XI) with a compound (VI) in the presence of a base.

This reaction is usually carried out in the presence of a solvent.

The solvent used in the reaction includes ethers such as THF, ethylene glycol dimethyl ether, and MTBE; aromatic hydrocarbons such as toluene, and xylene; halogenated hydrocarbons such as chlorobenzene; nitriles such as acetonitrile; acid amides such as DMF; sulfoxides such as dimethyl sulfoxide; ketones such as acetone, methyl ethyl ketone, and methyl isobutyl ketone; water; and mixtures thereof.

The base used in the reaction includes alkali metal carbonates such as sodium carbonate, potassium carbonate, and cesium carbonate; alkali metal hydroxides such as sodium hydroxide; and alkali metal hydrides such as sodium hydride.

The compound (VI) is usually used in an amount of 1 to 10 moles per mole of the compound (XI), and the base is usually used in an amount of 1 to 5 moles per mole of the compound (XI).

The reaction temperature ranges usually from −20° C. to 100° C., and the reaction time ranges usually from 0.1 to 24 hours.

After completion of the reaction, the compound (VIII) can be isolated by adding water to the reaction mixture, extracting the compound (VIII) with an organic solvent, and subjecting the organic layer to post-treatments such as drying and concentration. The compound (VIII) isolated may be further purified by operations such as chromatography, and recrystallization.

In some cases, the invented compound and the present amine compound have cis-trans isomers, i.e., a cis isomer and a trans isomer, relative to the carbon atom bonded to the carbon atom of the double bond, and in the present invention, a compound containing one of such active isomers or both of them in any ratio can be used as the invented compound or the present amine compound.

Specific examples of the invented compound are N-[2-fluoro-3-(2-propenyloxy)phenyl]methyl-quinoline-6-carboxamide, N-[3-(2-butenyloxy)-2-fluorophenyl]methyl-quinoline-6-carboxamide, N-[3-(3-butenyloxy)-2-fluorophenyl]methyl-quinoline-6-carboxamide, N-[2-fluoro-3-(2-pentenyloxy)phenyl]methyl-quinoline-6-carboxamide, N-[2-fluoro-3-(3-pentenyloxy)phenyl]methyl-quinoline-6-carboxamide, N-[2-fluoro-3-(4-pentenyloxy)phenyl]methyl-quinoline-6-carboxamide, N-[2-fluoro-3-(2-hexenyloxy)phenyl]methyl-quinoline-6-carboxamide, N-[2-fluoro-3-(3-hexenyloxy)phenyl]methyl-quinoline-6-carboxamide, N-[2-fluoro-3-(4-hexenyloxy)phenyl]methyl-quinoline-6-carboxamide, N-[2-fluoro-3-(5-hexenyloxy)phenyl]methyl-quinoline-6-carboxamide, N-[2-fluoro-3-(2-heptenyloxy)phenyl]methyl-quinoline-6-carboxamide, N-[2-fluoro-3-(3-heptenyloxy)phenyl]methyl-quinoline-6-carboxamide, N-[2-fluoro-3-(4-heptenyloxy)phenyl]methyl-quinoline-6-carboxamide, N-[2-fluoro-3-(5-heptenyloxy)phenyl]methyl-quinoline-6-carboxamide, N-[2-fluoro-3-(6-heptenyloxy)phenyl]methyl-quinoline-6-carboxamide, N-[2-fluoro-3-(2-octenyloxy)phenyl]methyl-quinoline-6-carboxamide, N-[2-fluoro-3-(3-octenyloxy)phenyl]methyl-quinoline-6-carboxamide, N-[2-fluoro-3-(4-octenyloxy)phenyl]methyl-quinoline-6-carboxamide, N-[2-fluoro-3-(5-octenyloxy)phenyl]methyl-quinoline-6-carboxamide, N-[2-fluoro-3-(6-octenyloxy)phenyl]methyl-quinoline-6-carboxamide,
N-[2-fluoro-3-(7-octenyloxy)phenyl]methyl-quinoline-6-carboxamide, N-[3-(2-propenyloxy)phenyl]methyl-quinoline-6-carboxamide,
N-[3-(2-butenyloxy)phenyl]methyl-quinoline-6-carboxamide, N-[3-(3-butenyloxy)phenyl]methyl-quinoline-6-carboxamide, N-[3-(2-pentenyloxy)phenyl]methyl-quinoline-6-carboxamide, N-[3-(3-pentenyloxy)phenyl]methyl-quinoline-6-carboxamide, N-[3-(4-pentenyloxy)phenyl]methyl-quinoline-6-carboxamide, N-[3-(2-hexenyloxy)phenyl]methyl-quinoline-6-carboxamide, N-[3-(3-hexenyloxy)phenyl]methyl-quinoline-6-carboxamide,
N-[3-(4-hexenyloxy)phenyl]methyl-quinoline-6-carboxamide, N-[3-(5-hexenyloxy)phenyl]methyl-quinoline-6-carboxamide, N-[3-(2-heptenyloxy)phenyl]methyl-quinoline-6-carboxamide, N-[3-(3-heptenyloxy)phenyl]methyl-quinoline-6-carboxamide, N-[3-(4-heptenyloxy)phenyl]methyl-quinoline-6-carboxamide, N-[3-(5-heptenyloxy)phenyl]methyl-quinoline-6-carboxamide, N-[3-(6-heptenyloxy)phenyl]methyl-quinoline-6-carboxamide, N-[3-(2-octenyloxy)phenyl]methyl-quinoline-6-carboxamide, N-[3-(3-octenyloxy)phenyl]methyl-quinoline-6-carboxamide, N-[3-(4-octenyloxy)phenyl]methyl-quinoline-6-carboxamide, N-[3-(5-octenyloxy)phenyl]methyl-quinoline-6-carboxamide,
N-[3-(6-octenyloxy)phenyl]methyl-quinoline-6-carboxamide, N-[3-(7-octenyloxy)phenyl]methyl-quinoline-6-carboxamide, N-[2-fluoro-3-(2-propynyloxy)phenyl]methyl-quinoline-6-carboxamide, N-[3-(2-butynyloxy)-2-fluorophenyl]methyl-quinoline-6-carboxamide, N-[3-(3-butynyloxy)-2-fluorophenyl]methyl-quinoline-6-carboxamide, N-[2-fluoro-3-(2-pentynyloxy)phenyl]methyl-quinoline-6-carboxamide, N-[2-fluoro-3-(3-pentynyloxy)phenyl]methyl-quinoline-6-carboxamide, N-[2-fluoro-3-(4-pentynyloxy)phenyl]methyl-quinoline-6-carboxamide, N-[2-fluoro-3-(2-hexynyloxy)phenyl]methyl-quinoline-6-carboxamide, N-[2-fluoro-3-(3-hexynyloxy)phenyl]methyl-quinoline-6-carboxamide, N-[2-fluoro-3-(4-hexynyloxy)phenyl]methyl-quinoline-6-carboxamide, N-[2-fluoro-3-(5-hexynyloxy)phenyl]methyl-quinoline-6-carboxamide, N-[2-fluoro-3-(2-heptynyloxy)phenyl]methyl-quinoline-6-carboxamide, N-[2-fluoro-3-(3-heptynyloxy)phenyl]methyl-quinoline-6-carboxamide, N-[2-fluoro-3-(4-heptynyloxy)phenyl]methyl-quinoline-6-carboxamide, N-[2-fluoro-3-(5-heptynyloxy)phenyl]methyl-quinoline-6-carboxamide, N-[2-fluoro-3-(6-heptynyloxy)phenyl]methyl-quinoline-6-carboxamide, N-[2-fluoro-3-(2-octynyloxy)phenyl]methyl-quinoline-6-carboxamide, N-[2-fluoro-3-(3-octynyloxy)phenyl]methyl-quinoline-6-carboxamide, N-[2-fluoro-3-(4-octynyloxy)phenyl]methyl-quinoline-6-carboxamide, N-[2-fluoro-3-(5-octynyloxy)phenyl]methyl-quinoline-6-carboxamide, N-[2-fluoro-3-(6-octynyloxy)phenyl]methyl-quinoline-6-carboxamide, N-[2-fluoro-3-(7-octynyloxy)phenyl]methyl-quinoline-6-carboxamide, N-[3-(2-propynyloxy)phenyl]methyl-quinoline-6-carboxamide,
N-[3-(2-butynyloxy)phenyl]methyl-quinoline-6-carboxamide, N-[3-(3-butynyloxy)phenyl]methyl-quinoline-6-carboxamide, N-[3-(2-pentynyloxy)phenyl]methyl-quinoline-6-carboxamide, N-[3-(3-pentynyloxy)phenyl]methyl-quinoline-6-carboxamide, N-[3-(4-pentynyloxy)phenyl]-methyl-quinoline-6-carboxamide, N-[3-(2-hexynyloxy)phenyl]methyl-quinoline-6-carboxamide, N-[3-(3-hexynyloxy)phenyl]methyl-quinoline-6-carboxamide, N-[3-(4-hexynyloxy)phenyl]methyl-quinoline-6-carboxamide,
N-[3-(5-hexynyloxy)phenyl]methyl-quinoline-6-carboxamide, N-[3-(2-heptynyloxy)phenyl]-methyl-quinoline-6-carboxamide, N-[3-(3-heptynyloxy)phenyl]methyl-quinoline-6-carboxamide, N-[3-(4-heptynyloxy)phenyl]methyl-quinoline-6-carboxamide, N-[3-(5-heptynyloxy)phenyl]methyl-quinoline-6-carboxamide, N-[3-(6-heptynyloxy)phenyl]methyl-quinoline-6-carboxamide, N-[3-(2-octynyloxy)phenyl]methyl-quinoline-6-carboxamide,
N-[3-(3-octynyloxy)phenyl]-methyl-quinoline-6-carboxamide, N-[3-(4-octynyloxy)phenyl]methyl-quinoline-6-carboxamide, N-[3-(5-octynyloxy)phenyl]methyl-quinoline-6-carboxamide, N-[3-(6-octynyloxy)phenyl]methyl-quinoline-6-carboxamide, N-[3-(7-octynyloxy)phenyl]methyl-quinoline-6-carboxamide, etc.

Specific examples of the present amine compound and its salts are
N-[2-fluoro-3-(2-propenyloxy)phenyl]methylamine,
N-[3-(2-butenyloxy)-2-fluorophenyl]methylamine,
N-[3-(3-butenyloxy)-2-fluorophenyl]methylamine,
N-[2-fluoro-3-(2-pentenyloxy)phenyl]methylamine,
N-[2-fluoro-3-(3-pentenyloxy)phenyl]methylamine,
N-[2-fluoro-3-(4-pentenyloxy)phenyl]methylamine,
N-[2-fluoro-3-(2-hexenyloxy)phenyl]methylamine,
N-[2-fluoro-3-(3-hexenyloxy)phenyl]methylamine,
N-[2-fluoro-3-(4-hexenyloxy)phenyl]methylamine,
N-[2-fluoro-3-(5-hexenyloxy)phenyl]methylamine,
N-[2-fluoro-3-(2-heptenyloxy)phenyl]methylamine,
N-[2-fluoro-3-(3-heptenyloxy)phenyl]methylamine,
N-[2-fluoro-3-(4-heptenyloxy)phenyl]methylamine,
N-[2-fluoro-3-(5-heptenyloxy)phenyl]methylamine,
N-[2-fluoro-3-(6-heptenyloxy)phenyl]methylamine,
N-[2-fluoro-3-(2-octenyloxy)phenyl]methylamine,
N-[2-fluoro-3-(3-octenyloxy)phenyl]methylamine,
N-[2-fluoro-3-(4-octenyloxy)phenyl]methylamine,
N-[2-fluoro-3-(5-octenyloxy)phenyl]methylamine,
N-[2-fluoro-3-(6-octenyloxy)phenyl]methylamine,
N-[2-fluoro-3-(7-octenyloxy)phenyl]methylamine,
N-[3-(2-propenyloxy)phenyl]methylamine,
N-[3-(2-butenyloxy)phenyl]methylamine,
N-[3-(3-butenyloxy)phenyl]methylamine,
N-[3-(2-pentenyloxy)phenyl]methylamine,
N-[3-(3-pentenyloxy)phenyl]methylamine,
N-[3-(4-pentenyloxy)phenyl]methylamine,
N-[3-(2-hexenyloxy)phenyl]methylamine,
N-[3-(3-hexenyloxy)phenyl]methylamine,
N-[3-(4-hexenyloxy)phenyl]methylamine,
N-[3-(5-hexenyloxy)phenyl]methylamine,
N-[3-(2-heptenyloxy)phenyl]methylamine,
N-[3-(3-heptenyloxy)phenyl]methylamine,
N-[3-(4-heptenyloxy)phenyl]methylamine,
N-[3-(5-heptenyloxy)phenyl]methylamine,
N-[3-(6-heptenyloxy)phenyl]methylamine,
N-[3-(2-octenyloxy)phenyl]methylamine,
N-[3-(3-octenyloxy)phenyl]methylamine,
N-[3-(4-octenyloxy)phenyl]methylamine,
N-[3-(5-octenyloxy)phenyl]methylamine,
N-[3-(6-octenyloxy)phenyl]methylamine,
N-[3-(7-octenyloxy)phenyl]methylamine,
N-[2-fluoro-3-(2-propynyloxy)phenyl]methylamine,
N-[3-(2-butynyloxy)-2-fluorophenyl]methylamine,
N-[3-(3-butynyloxy)-2-fluorophenyl]methylamine,
N-[2-fluoro-3-(2-pentynyloxy)phenyl]methylamine,
N-[2-fluoro-3-(3-pentynyloxy)phenyl]methylamine,
N-[2-fluoro-3-(4-pentynyloxy)phenyl]methylamine,
N-[2-fluoro-3-(2-hexynyloxy)phenyl]methylamine,
N-[2-fluoro-3-(3-hexynyloxy)phenyl]methylamine,
N-[2-fluoro-3-(4-hexynyloxy)phenyl]methylamine,
N-[2-fluoro-3-(5-hexynyloxy)phenyl]methylamine,
N-[2-fluoro-3-(2-heptynyloxy)phenyl]methylamine,
N-[2-fluoro-3-(3-heptynyloxy)phenyl]methylamine,
N-[2-fluoro-3-(4-heptynyloxy)phenyl]methylamine,
N-[2-fluoro-3-(5-heptynyloxy)phenyl]methylamine,
N-[2-fluoro-3-(6-heptynyloxy)phenyl]methylamine,
N-[2-fluoro-3-(2-octynyloxy)phenyl]methylamine,
N-[2-fluoro-3-(3-octynyloxy)phenyl]methylamine,
N-[2-fluoro-3-(4-octynyloxy)phenyl]methylamine,
N-[2-fluoro-3-(5-octynyloxy)phenyl]methylamine,
N-[2-fluoro-3-(6-octynyloxy)phenyl]methylamine,
N-[2-fluoro-3-(7-octynyloxy)phenyl]methylamine,
N-[3-(2-propynyloxy)phenyl]methylamine,
N-[3-(2-butynyloxy)phenyl]methylamine,
N-[3-(3-butynyloxy)phenyl]methylamine,
N-[3-(2-pentynyloxy)phenyl]methylamine,
N-[3-(3-pentynyloxy)phenyl]methylamine,
N-[3-(4-pentynyloxy)phenyl]methylamine,
N-[3-(2-hexynyloxy)phenyl]methylamine,
N-[3-(3-hexynyloxy)phenyl]methylamine,
N-[3-(4-hexynyloxy)phenyl]methylamine,
N-[3-(5-hexynyloxy)phenyl]methylamine,
N-[3-(2-heptynyloxy)phenyl]methylamine,
N-[3-(3-heptynyloxy)phenyl]methylamine,
N-[3-(4-heptynyloxy)phenyl]methylamine,
N-[3-(5-heptynyloxy)phenyl]methylamine,
N-[3-(6-heptynyloxy)phenyl]methylamine,
N-[3-(2-octynyloxy)phenyl]methylamine,
N-[3-(3-octynyloxy)phenyl]methylamine,
N-[3-(4-octynyloxy)phenyl]methylamine,
N-[3-(5-octynyloxy)phenyl]methylamine,
N-[3-(6-octynyloxy)phenyl]methylamine,
N-[3-(7-octynyloxy)phenyl]methylamine, and their salts (inorganic acid salts such as hydrochloride, hydrobromide, and sulfate; and organic acid salts such as acetate, trifluoroacetate, formate, oxalate, methanesulfonate, and p-toluenesulfonate).

Although the invented controlling agent may be composed of only the invented compound, the invented compound is used usually after having been formulated into any of formulations such as wettable powders, water dispersible granules, flowable concentrates, granules, dry flowable concentrates, emulsifiable concentrates, aqueous liquid formulations, oil formulations, smoking formulations, aerosols, and microcapsules by mixing with a carrier (e.g. a solid, liquid or gaseous carrier), a surfactant and other auxiliaries for formulation, such as adhesive agent, dispersant, and stabilizer. Such formulations contain the invented compound in a proportion of usually 0.1 to 99% by weight, preferably 0.2 to 90% by weight.

Solid carriers used for formulation include, for example, fine powders or particles of clays (e.g., kaolin, diatomaceous earth, synthetic hydrous silicon oxide, agalmatolite clay, bentonite, acid clay, and talc), and other inorganic minerals (e.g., sericite, quartz powder, sulfur powder, activated carbon, calcium carbonate, and hydrated silica), liquid carriers include, for example, water, alcohols (e.g., methanol, and ethanol), ketones (e.g., acetone, and methyl ethyl ketone), aromatic hydrocarbons (e.g., benzene, toluene, xylene, ethylbenzene, and methylnaphthalene), aliphatic or alicyclic hydrocarbons (e.g., n-hexane, cyclohexanone, and kerosene), esters (e.g., ethyl acetate, and butyl acetate), nitriles (e.g., acetonitrile, and isobutyronitrile), ethers (e.g., dioxane, and diisopropyl ether), acid amides (e.g., dimethylformamide, and dimethylacetamide), and halogenated hydrocarbons (e.g., dichloroethane, trichloroethylene, and carbon tetrachloride).

The surfactant includes, for example, alkyl sulfates, alkylsulfonates, alkylarylsulfonates, alkyl aryl ethers and their polyoxyethylenated products, polyoxyethylene glycol ethers, polyhydric alcohol esters and sugar alcohol derivatives.

Other auxiliaries for formulation include adhesive agents, dispersants, thickening agents, wetting agents, diluents and antioxidants. Specific examples thereof are casein, gelatin, polysaccharides (e.g. starch, gum arabic, cellulose derivative and alginic acid), lignin derivatives, bentonite, saccharides, synthetic water-soluble polymers [e.g. poly(vinyl alcohols, poly(vinylpyrrolidone)s and poly(acrylic acid)s], PAP (acidic isopropyl phosphate), BHT (2,6-di-tert-butyl-4-methylphenol), BHA (a mixture of 2-tert-butyl-4-methoxyphenol and 3-tert-butyl-4-methoxyphenol), vegetable oils, mineral oils, fatty acids, and their esters.

A method for applying the invented controlling agent in order to control plant diseases is not particularly limited. The method includes treatment of plants, such as foliage application; treatment of a plant cultivation area, such as soil treatment; and treatment of seeds, such as seed disinfection.

The invented controlling agent may be used in admixture with other fungicides, insecticides, acaricides, nematicides, herbicides, plant growth regulators, fertilizers or soil conditioners. It is also possible to use the invented controlling agent in combination with such other chemicals without mixing with them.

Such other fungicides include azole fungicidal compounds such as propiconazole, prothioconazole, triadimenol, prochloraz, penconazole, tebuconazole, flusilazole, diniconazole, bromuconazole, epoxiconazole, difenconazole, cyproconazole, metconazole, triflumizole, tetraconazole, myclobutanil, fenbuconazole, hexaconazole, fluquinconazole, triticonazole, bitertanol, imazalil, and flutriafol; cyclic amine fungicidal compounds such as fenpropimorph, tridemorph, and fenpropidin; benzimidazole fungicidal compounds such as carbendazim, benomyl, thiabendazole, and thiophanate-methyl; procymidone, cyprodinil, pyrimethanil, diethofencarb, thiuram, fluazinam, mancozeb, iprodione, vinclozolin, chlorothalonil, captan, mepanipyrim, fenpiclonil, fludioxonil, dichlofluanid, folpet, kresoxim-methyl, azoxystrobin, trifloxystrobin, fluoxastrobin, picoxystrobin, pyraclostrobin, dimoxystrobin, pyribencarb, spiroxamine, quinoxyfen, fenhexamid, famoxadone, fenamidone, zoxamide, ethaboxam, amisulbrom, iprovalicarb, benthiavalicarb, cyazofamid, mandipropamid, boscalid, penthiopyrad, metrafenone, fluopyram, bixafen, cyflufenamid and proquinazid.

While the applying dosage of the invented controlling agent is varied depending on weather conditions, formulation, when, where and how the invented controlling agent is applied, diseases to be controlled, crop plants to be protected, etc., it is usually 1 to 500 g, preferably 2 to 200 g, (in terms of the invented compound in the invented controlling agent), per 10 ares. When the invented controlling agent is an emulsifiable concentrate, wettable powder, suspension concentrate or the like, it is usually applied after having been diluted with water. In this case, the concentration of the invented compound after the dilution is usually 0.0005 to 2% by weight, preferably 0.005 to 1% by weight. When the invented controlling agent is a powder, granules or the like, it is applied as it is without dilution. When the invented controlling agent is applied to seeds, its applying dosage is usually 0.001 to 100 g, preferably 0.01 to 50 g, (in terms of the invented compound in the invented controlling agent), per kilogram of the seeds.

The invented controlling agent can be used as a composition for controlling plant diseases in crop lands such as upland field, paddy field, lawn and turf, and orchard. The present controller can control plant diseases in crop lands where the following “crops” or the like are cultivated.

Field crops: corn, rice, wheat, barley, rye, oat, sorghum, cotton, soybean, peanut, buckwheat, beet, rape, sunflower, sugar cane, tobacco, etc.

Vegetables: Solanaceae (e.g. eggplant, tomato, green pepper, pepper and potato), Cucurbotaceae (e.g. cucumber, pumpkin, zucchini, watermelon and melon), Cruciferae (e.g. Japanese radish, turnip, horseradish, kohlrabi, Chinese cabbage, cabbage, leaf mustard, broccoli and cauliflower), Compositae (e.g. edible burdock, garland chrysanthemum, globe artichoke and lettuce), Liliacede (e.g. Welsh onion, onion, garlic and asparagus), Umbelliferae (e.g. carrot, parsley, celery and Pstinaca), Chenopodiales (e.g. spinach and chard), Lamiaceae (e.g. perilla, mint and basil), strawberry, sweet potato, Chinese yam, taro, etc.

Flowers and ornament plants.

Ornamental foliage plants.

Fruit trees: pomaceous fruits (e.g. apple, pear, Japanese pear, Chinese quince and quince), stone fruits (e.g. peach, plum, nectarine, Japanese apricot, cherry, apricot and prune), citrus fruits (e.g. Satsuma mandarin, orange, lemon, lime and grapefruit), nut trees (e.g. chestnut, walnut, hazel, almond, pistachio, cashew nut and macadamia nut), berries (e.g. blueberry, cranberry, blackberry and raspberry), grape, Japanese persimmon, olive, loquat, banana, coffee, date palm, coconut palm, etc.

Trees other than fruit trees: tea, mulberry, flowering trees and shrubs, street trees (Japanese ash, birch, flowering dogwood, blue gum, ginkgo, lilac, maple, oak, poplar, Chinese redbud, Formosa sweet gum, plane trees, zelkova, Japanese arborvitae, fir, Japanese hemlock, needle juniper, pine, Japanese spruce and Japanese yew).

The above-mentioned “crops” also include crops having resistance to herbicides such as HPPD inhibitors (e.g. isoxaflutole), ALS inhibitor (e.g. imazethapyr and thifensulfuron-methyl), EPSP synthetase inhibitors, glutamine synthetase inhibitors, bromoxynil, dicamba, etc. which has been imparted by a classic breeding method or a genetic recombination technology.

Examples of the “crops” having the resistance imparted by the classic breeding method include Clearfield Canola® resistant to imidazolinone herbicides (e.g. imazethapyr) and STS soybean resistant to sulfonylurea ALS inhibition type herbicides (e.g. thifensulfuron-methyl). Examples of crops having the resistance imparted by the genetic recombination technology include corn cultivars resistant to glyphosate and glufosinato, which are already on the market under the trade names of RoundupReady®, RoundupReady 2® and LibertyLink®.

The above-mentioned “crops” also include crops which a genetic recombination technology has enabled to synthesize a selective toxin known in the case of, for example, Bacillus.

Examples of toxins produced in such genetically modified plants include insecticidal proteins derived from Bacillus cereus and Bacillus popilliae; insecticidal proteins such as δ-endotoxins (e.g. Cry1Ab, Cry1Ac, Cry1F, Cry1Fa2, Cry2Ab, Cry3A, Cry3Bb1 and Cry9C), VIP1, VIP2, VIP3, and VIP3A, which are derived from Bacillus thuringiensis; toxins derived from nematodes; toxins produced by animals, such as scorpion toxin, spider toxin, bee toxin, and insect-specific neurotoxins; filamentous fungi toxins; plant lectins; agglutinin; protease inhibitors such as trypsin inhibitors, serine protease inhibitors, patatin, cystatin, and papain inhibitors; ribosome-inactivating proteins (RIP) such as ricin, corn-RIP, abrin, rufin, sapolin, and priodin; steroid metabolic enzymes such as 3-hydroxysteroid oxidase, ecdysteroid-UDP-glucosyltransferase, and cholesterol oxidase; ecdysone inhibitors; HMG-COA reductase; ion channel inhibitors such as sodium channel inhibitors, and calcium channel inhibitors; juvenile hormone esterase; diuretic hormone receptors; stilbene synthetase; bibenzyl synthetase; chitinase; and glucanase.

The toxins produced in such genetically modified crops also include hybrid toxins, partly deficient toxins and modified toxins of insecticidal proteins such as δ-endotoxin proteins (e.g. Cry1Ab, Cry1Ac, Cry1F, Cry1Fa2, Cry2Ab, Cry3A, Cry3Bb1 and Cry9C), VIP1, VIP2, VIP3, and VIP3A. The hybrid toxins are produced by a novel combination of the different domains of such a protein by adopting a recombination technology. As the partly deficient toxin, Cry1Ab deficient in a part of the amino acid sequence is known. In the modified toxins, one or more amino acids of a natural toxin have been replaced.

Examples of such toxins and genetically modified plants capable of synthesizing such toxins are described in EP-A-0 374 753, WO 93/07278, WO95/34656, EP-A-0 427 529, EP-A-451 878, WO 03/052073, etc.

The toxins contained in such genetically modified plants impart resistance to insect pests of Coleoptera, insect pests of Diptera and insect pests of Lepidoptera to the plants.

Genetically modified plants containing one or more insecticidal insect-resistant genes and capable of producing one or more toxins have already been known, and some of them are on the market. Examples of such genetically modified plants include YieldGard® (a corn cultivar capable of producing Cry1Ab toxin), YieldGard Rootworm® (a corn cultivar capable of producing Cry3Bb1 toxin), YieldGard Plus® (a corn cultivar capable of producing Cry1Ab and Cry3Bb1 toxins), Herculex I® (a corn cultivar capable of producing phosphinotrysin N-acetyltransferase (PAT) for imparting resistance to Cry1Fa2 toxin and Glyfosinate), NuCOTN33B (a cotton cultivar capable of producing Cry1Ac toxin), Bollgard I® (a cotton cultivar capable of producing Cry1Ac toxin), Bollgard II® (a cotton cultivar capable of producing Cry1Ac and Cry2Ab toxins), VIPCOT® (a cotton cultivar capable of producing VIP toxin), NewLeaf® (a potato cultivar capable of producing Cry3A toxin), NatureGard®, Agrisure®, CB Advantage (Bt11 corn borer (CB) properties), and Protecta®.

The above-mentioned “crops” also include crops having an ability to produce an anti-pathogenic substance having selective action which has been imparted by a gene recombination technology.

As examples of the anti-pathogenic substance, PR proteins and the like are known (PRPs, EP-A-0 392 225). Such anti-pathogenic substances and genetically modified plants capable of producing them are described in EP-A-0 392 225, WO 95/33818, EP-A-0 353 191, etc.

Examples of such anti-pathogenic substances produced by the genetically modified plants include ion channel inhibitors such as sodium channel inhibitors, and calcium channel inhibitors (for example, KP1, KP4 and KP6 toxins produced by viruses are known); stilbene synthases; bibenzyl synthases; chitinase; glucanase; PR proteins; and anti-pathogenic substances produced by microorganisms, such as peptide antibiotics, antibiotics having a heterocyclic ring, and protein factors concerned in resistance to plant diseases (which are called plant-disease-resistant genes and are described in WO 03/000906).

The above-mentioned “crops” also include crops having two or more properties relating to the above-mentioned herbicide resistance, insect pest resistance, disease resistance and the like, which have been imparted by a classic breeding technique or a genetic recombination technology; and crops having two or more properties derived from parents which have been imparted by mating between genetically modified plants having the same or different properties.

Plant diseases controllable by the present invention include, for example, fungal diseases. More particularly, the diseases described below can be exemplified as the plant diseases. The plant diseases are not limited to them.

The invented controlling method is usually practiced by applying the invented controlling agent by the above-mentioned method for applying the invented controlling agent.

Blast (Magnaporthe grisea), Helminthosporium leaf spot (Cochliobolus miyabeanus), sheath blight (Rhizoctonia solani) and “Bakanae” disease (Gibberella fujikuroi) of rice; powdery mildew (Erisiphe graminis), scab (Fusarium graminearum, F. avenacerum, F. culmorum, Microdochium nivale), rust (Puccinia striiformis, P. graminis, P. recondite, P. hordei), snow blight (Typhula sp., Micronectriella nivalis), loose smut (Ustilago tritici, U. nude), bunt (Tilletia caries), eyespot (Pseudocercosporella herpotrichoides), scald (Rhynchosporium secalis), leaf blight (Septoria tritici), glume blotch (Leptosphaeria nodorum), net blotch (Pyrenophora teres Drechaler) and take-all (Gaeumannomyces graminis) of barley, wheat, oats and rye; melanose (Diaporthe citri), scab (Elsinoe fawcetti) and penicillium rot (Penicillium digitatum, P. italicum) of citrus; blossom blight (Monilinia mali), canker (Valsa ceratosperma), powdery mildew (Podosphaera leucotricha), Alternaria leaf spot (Alternaria altenate apple pathotype), scab (Venturia inaqualis) and anthracnose (Glomerella cingulata) of apple; scab (Venturia nashicola, V. pirina), black spot (Alternaria alternate Japanese pear pathotype) and rust (Gymnosporangium haraeanum) of pear; brown rot (Monilinia fructicola), scab (Cladosporium carpophilum) and Phomopsis rot (Phomopsis sp.) of peach;

anthracnose (Elsinoe ampelina), ripe rot (Glomerella cingulata), powdery mildew (Uncinula necator), rust (Phakopsora ampelopsidis), black rot (Guignardia bidwellii) and downy mildew (Plasmopara viticola) of grape; anthracnose (Gloeosporium kaki) and leaf spot (Cercospora Mycosphaerella nawae) of Japanese persimmon; anthracnose (Colletotrichum lagenarium), powdery mildew (Sphaerotheca fuliginea), gummy stem blight (Mycosphaerella melonis), stem rot (Fusarium oxyspoxum), downy mildew (Pseudoperonospora cubensis), Phytophthora rot (Phytophthora sp.) and seedling blight (Phthium sp.) of melons and cucumber; early blight (Alternaria solani), leaf mold (Cladosporium fulvum) and late blight (Phytophthora infestans) of tomato; brown spot (Phomopsis vexans) and powdery mildew (Erisiphe cichoracearum) of eggplant; alternaria leaf spot (Alternaria japonica) and white spot (Cercosporella brassicae) of vegetables of Crusiferae; Welsh onion rust (Puccinia allii); purple stain (Cercospora kikuchii), Sphaceloma scab (Elsinoe glycines), pod and stem blight (Diaporthe phaseolorum var. sojae) and rust (Phakopsora pachyrhizi) of soybean; kidney bean anthracnose (Colletotrichum lindemthianum); leaf spot (Cercospora personata), leaf spot (Cercospora arachidicola) and southern blight (Sclerotium rolfsii) of peanut; pea powdery mildew (Erisiphe pisi); early blight (Alternaria solani), late blight (Phytophthora infestans) and Verticillium wilt (Verticillium albo-atrum, V. dahliae, V. nigrescens) of potato; strawberry powdery mildew (Sphaerotheca humuli); net blister blight (Exobasidium reticulatum), white scab (Elsinoe leucospila), zonate leaf spot (Pestalotiopsis sp.) and anthracnose (Colletotrichum theae-sinensis) of tea plant; brown spot (Alternaria longipes), powdery mildew (Erysiphe cichoracearum), anthracnose (Colletotrichum tabacum), downy mildew (Peronospora tabacina), Phytophthora rot (Phytophthora nicotianae) of tobacco;
leaf spot (Cercospora beticola), foliage blight (Thanatephorus cucumeris) and root rot (Thanatephorus cucumeris) of beet; black spot (Diplocarpon rosae) and powdery mildew (Sphaerotheca pannosa) of rose; leaf blight (Septoria chrysanthemi-indici) and white rust (Puccinia horiana) of chrysanthemum; Botrytis diseases (Botrytis cinerea, B. byssoidea, B. squamosa), gray mold neck rot (Botrytis alli) and Small sclerotial neck rot (Botrytis squamosa) of onion; gray mold (Botrytis cinerea) and stem rot (Sclerotinia sclerotiorum) of various crops; Alternaria leaf spot (Alternaria brassicicola) of Japanese radish; dollar spot (Sclerotinia homeocarpa), brown patch and large patch (Rhizoctonia solani) of turf grass; and Sigatoka diseases (Mycosphaerella fijiensis, Mycosphaerella musicola, Pseudocercospora musae) of banana.

### EXAMPLES

The present invention will be explained in more detail with reference to preparation examples, formulation examples and test examples, which should not be construed as limiting the invention. All parts are by weight.

### Preparation Example 1 of the Invented Compound

To a mixture of 0.17 g of N-(2-fluoro-3-hydroxyphenyl)methyl-quinoline-6-carboxamide, 0.09 g of 3-bromo-1-propene and 3 ml of DMF was added 0.25 g of cesium carbonate, and the resulting mixture was stirred at room temperature for 12 hours. Water was added to the reaction mixture and the solid precipitated was collected by filtration and washed successively with an aqueous sodium hydroxide solution, water and hexane to obtain 0.12 g of N-[2-fluoro-3-(2-propenyloxy)phenyl]methyl-quinoline-6-carboxamide (hereinafter referred to as the invented compound (1)).

### The Invented Compound (1)

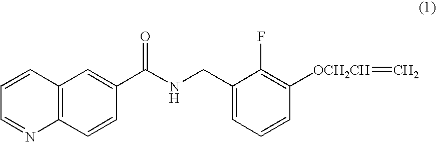

¹H-NMR (CDCl₃) δ: 4.61 (2H, d, J=3.2 Hz), 4.76 (2H, d, J=4.1 Hz), 5.32 (1H, d, J=10.5 Hz), 5.44 (1H, d, J=16.1 Hz), 6.03-6.11 (1H, m), 6.72 (1H, br s), 6.92-7.06 (3H, m), 7.45-7.49 (1H, m), 8.05-8.07 (1H, m), 8.15 (1H, d, J=8.3 Hz), 8.23 (1H, d, J=8.0 Hz), 8.31 (1H, s), 8.98 (1H, s).

### Preparation Example 2 of the Invented Compound

To a mixture of 0.30 g of N-(2-fluoro-3-hydroxyphenyl)methyl-quinoline-6-carboxamide, 54 mg of sodium hydride (55% dispersion in oil) and 5 ml of DMF was added 0.27 g of 4-bromo-1-butene, and the resulting mixture was stirred at 100° C. for 7 hours. Then, the reaction mixture was allowed to cool to about room temperature and water was added thereto, followed by extraction with ethyl acetate. The organic layer was washed with saturated aqueous sodium chloride solution, dried over magnesium sulfate and then concentrated under reduced pressure. The residue was subjected to thin-layer chromatography to obtain 72 mg of N-[3-(3-butenyloxy)-2-fluorophenyl]methyl-quinoline-6-carboxamide (hereinafter referred to as the invented compound (2)).

### The Invented Compound (2)

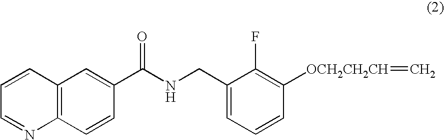

¹H-NMR (CDCl₃) δ: 8.93 (1H, s), 8.29 (1H, s), 8.13 (1H, d, J=8.0 Hz), 8.06 (2H, s), 7.41 (1H, dd, J=8.3, 4.1 Hz), 7.16 (1H, s), 7.00-6.98 (2H, m), 6.90-6.86 (1H, m), 5.94-5.84 (1H, m), 5.17 (1H, d, J=17.1 Hz), 5.11 (1H, d, J=10.2 Hz), 4.72 (2H, d, J=5.6 Hz), 4.04 (2H, t, J=6.7 Hz), 2.58-2.53 (2H, m).

### Preparation Example 3 of the Invented Compound

To a mixture of 0.30 g of N-(2-fluoro-3-hydroxyphenyl)methyl-quinoline-6-carboxamide, 0.17 g of 5-bromo-1-pentene and 5 ml of DMF was added 0.43 g of cesium carbonate, and the resulting mixture was stirred at room temperature for 12 hours. Then, water was added to the reaction mixture, followed by extraction with ethyl acetate. The organic layer was washed with saturated aqueous sodium chloride solution, dried over magnesium sulfate and then concentrated under reduced pressure. The residue was subjected to silica gel chromatography to obtain 0.26 of N-[2-fluoro-3-(4-pentenyloxy)phenyl]methyl-quinoline-6-carboxamide (hereinafter referred to as the invented compound (3)).

### The Invented Compound (3)

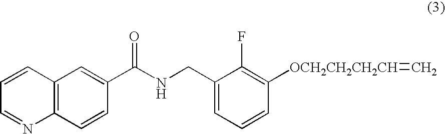

¹H-NMR (CDCl₃) δ: 8.98 (1H, dd, J=4.1, 1.7 Hz), 8.31 (1H, d, J=2.0 Hz), 8.23 (1H, dd, J=8.7, 1.1 Hz), 8.14 (1H, d, J=8.8 Hz), 8.06 (1H, dd, J=8.9, 2.1 Hz), 7.46 (1H, dd, J=8.3, 4.4 Hz), 7.06-6.99 (2H, m), 6.94-6.90 (1H, m), 6.71 (1H, s), 5.90-5.80 (1H, m), 5.10-5.04 (1H, m), 5.03-4.99 (1H, m), 4.76 (2H, dd, J=5.9, 1.0 Hz), 4.04 (2H, t, J=6.5 Hz), 2.29-2.24 (2H, m), 1.97-1.89 (2H, m).

### Preparation Example 4 of the Invented Compound

To a mixture of 0.30 g of N-(2-fluoro-3-hydroxyphenyl)methyl-quinoline-6-carboxamide, 0.18 g of 6-bromo-1-hexene and 5 ml of DMF was added 0.43 g of cesium carbonate, and the resulting mixture was stirred at room temperature for 12 hours. Then, water was added to the reaction mixture, followed by extraction with ethyl acetate. The organic layer was washed with saturated aqueous sodium chloride solution, dried over magnesium sulfate and then concentrated under reduced pressure. After the concentration, the residue was subjected to silica gel chromatography to obtain 0.30 of N-[2-fluoro-3-(5-hexenyloxy)phenyl]methyl-quinoline-6-carboxamide (hereinafter referred to as the invented compound (4)).

### The Invented Compound (4)

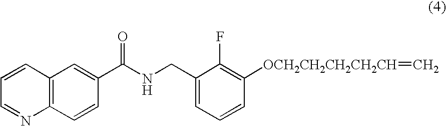

¹H-NMR (CDCl₃) δ: 8.98 (1H, dd, J=4.1, 1.7 Hz), 8.31 (1H, d, J=1.7 Hz), 8.23 (1H, d, J=7.8 Hz), 8.14 (1H, d, J=8.8 Hz), 8.06 (1H, dd, J=8.8, 2.0 Hz), 7.47 (1H, dd, J=8.4, 4.3 Hz), 7.07-6.99 (2H, m), 6.94-6.90 (1H, m), 6.70 (1H, s), 5.88-5.78 (1H, m), 5.06-5.01 (1H, m), 4.99-4.96 (1H, m), 4.76 (2H, d, J=5.9 Hz), 4.04 (2H, t, J=6.5 Hz), 2.17-2.11 (2H, m), 1.88-1.81 (2H, m), 1.63-1.56 (2H, m).

### Preparation Example 5 of the Invented Compound

To a mixture of 0.30 g of N-(2-fluoro-3-hydroxyphenyl)methyl-quinoline-6-carboxamide, 0.22 g of 7-bromo-1-heptene and 4 ml of DMF was added 0.50 g of cesium carbonate, and the resulting mixture was stirred at room temperature for 12 hours. Water was added to the reaction mixture and the solid precipitated was collected by filtration and washed with water and then hexane to obtain 0.32 g of N-[2-fluoro-3-(6-heptenyloxy)phenyl]methyl-quinoline-6-carboxamide (hereinafter referred to as the invented compound (5)).

### The Invented Compound (5)

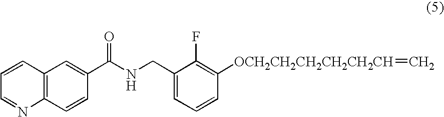

¹H-NMR (CDCl₃) δ: 9.00-8.98 (1H, m), 8.31 (1H, d, J=1.5 Hz), 8.24 (1H, d, J=8.5 Hz), 8.15 (1H, d, J=8.8 Hz), 8.06 (1H, dd, J=8.8, 1.7 Hz), 7.47 (1H, dd, J=8.3, 4.1 Hz), 7.07-7.00 (2H, m), 6.95-6.90 (1H, m), 6.66 (1H, s), 5.87-5.77 (1H, m), 5.03-4.99 (1H, m), 4.96-4.94 (1H, m), 4.76 (2H, d, J=5.9 Hz), 4.03 (2H, t, J=6.6 Hz), 2.11-2.07 (2H, m), 1.87-1.80 (2H, m), 1.54-1.44 (4H, m).

### Preparation Example 6 of the Invented Compound

To a mixture of 0.30 g of N-(2-fluoro-3-hydroxyphenyl)methyl-quinoline-6-carboxamide, 0.23 g of 8-bromo-1-octene and 4 ml of DMF was added 0.50 g of cesium carbonate, and the resulting mixture was stirred at room temperature for 12 hours. Water was added to the reaction mixture and the solid precipitated was collected by filtration and washed with water and then hexane to obtain 0.32 g of N-[2-fluoro-3-(7-octenyloxy)phenyl]methyl-quinoline-6-carboxamide (hereinafter referred to as the invented compound (6)).

### The Invented Compound (6)

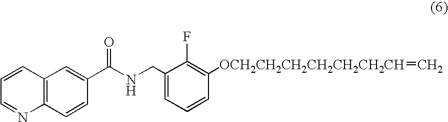

¹H-NMR (CDCl₃) δ: 9.00-8.98 (1H, m), 8.31 (1H, d, J=1.7 Hz), 8.24 (1H, d, J=8.0 Hz), 8.15 (1H, d, J=8.5 Hz), 8.06 (1H, dd, J=8.8, 2.0 Hz), 7.47 (1H, dd, J=8.3, 4.1 Hz), 7.07-7.00 (2H, m), 6.94-6.90 (1H, m), 6.66 (1H, s), 5.86-5.76 (1H, m), 5.00 (1H, dd, J=17.2, 1.6 Hz), 4.94 (1H, d, J=10.0 Hz), 4.76 (2H, d, J=5.9 Hz), 4.03 (2H, t, J=6.6 Hz), 2.09-2.04 (2H, m), 1.86-1.79 (2H, m), 1.52-1.35 (6H, m).

### Preparation Example 7 of the Invented Compound

To a mixture of 0.50 g of N-(2-fluoro-3-hydroxyphenyl)methyl-quinoline-6-carboxamide, 0.34 g of potassium carbonate and 5 ml of DMF was added 0.34 g of 4-bromo-2-butene, and the resulting mixture was stirred at room temperature for 8 hours. Then, water was added to the reaction mixture, followed by extraction with ethyl acetate. The organic layer was washed with saturated aqueous sodium chloride solution, dried over magnesium sulfate and then concentrated under reduced pressure. The residue was subjected to column chromatography to obtain 0.33 of N-[3-(2-butenyloxy)-2-fluorophenyl]methyl-quinoline-6-carboxamide (hereinafter referred to as the invented compound (7)).

### The Invented Compound (7)

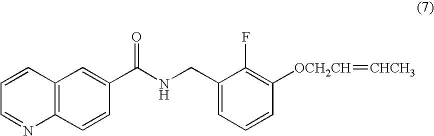

¹H-NMR (CDCl₃) δ: 1.74-1.77 (3.0H, m), 4.53 (1.8H, d, J=6.1 Hz), 4.68 (0.2H, d, J=6.1 Hz), 4.76 (2.0H, d, J=5.9 Hz), 5.71-5.78 (1.1H, m), 5.84-5.93 (0.9H, m), 6.66 (1.0H, s), 6.91-6.97 (1.0H, m), 7.01-7.07 (2.0H, m), 7.47 (1.0H, dd, J=8.3, 4.1 Hz), 8.06 (1.0H, dd, J=8.8, 2.0 Hz), 8.15 (1.0H, d, J=8.8 Hz), 8.24 (1.0H, d, J=8.3 Hz), 8.31 (1.0H, d, J=2.0 Hz), 8.99 (1.0H, dd, J=4.1, 1.7 Hz).

### Preparation Example 8 of the Invented Compound

To a mixture of 0.30 g of N-(2-fluoro-3-hydroxyphenyl)methyl-quinoline-6-carboxamide, 0.21 g of potassium carbonate and 5 ml of DMF was added 0.18 g of 1-bromo-3-pentene, and the resulting mixture was stirred at room temperature for 8 hours. Then, water was added to the reaction mixture, followed by extraction with ethyl acetate. The organic layer was washed with saturated aqueous sodium chloride solution, dried over magnesium sulfate and then concentrated under reduced pressure. The residue was subjected to thin-layer chromatography to obtain 0.27 of N-[3-(2-pentenyloxy)-2-fluorophenyl]methyl-quinoline-6-carboxamide (hereinafter referred to as the invented compound (8)).

### The Invented Compound (8)

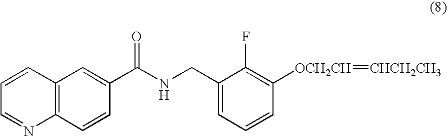

¹H-NMR (CDCl₃) δ: 1.00-1.04 (3.0H, m), 2.09-2.17 (2.0H, m), 4.55 (1.2H, d, J=6.1 Hz), 4.67 (0.8H, d, J=5.4 Hz), 4.76 (2.0H, d, J=5.9 Hz), 5.64-5.75 (1.4H, m), 5.87-5.89 (0.6H, m), 6.65 (1.0H, s), 6.92-6.97 (1.0H, m), 7.02-7.05 (2.0H, m), 7.47 (1.0H, dd, J=8.3, 4.1 Hz), 8.06 (1.0H, dd, J=8.8, 2.2 Hz), 8.15 (1.0H, d, J=8.8 Hz), 8.24 (1.0H, d, J=8.3 Hz), 8.31 (1.0H, d, J=2.0 Hz), 8.98-9.00 (1.0H, m).

### Preparation Example 9 of the Invented Compound

To a mixture of 0.19 g of N-(3-hydroxyphenyl)methyl-quinoline-6-carboxamide, 0.12 g of 3-bromo-1-propene and 3 ml of DMF was added 0.25 g of cesium carbonate, and the resulting mixture was stirred at room temperature for 12 hours. Water was added to the reaction mixture and the solid precipitated was collected by filtration and washed successively with aqueous sodium hydroxide solution, water and hexane to obtain 0.13 g of N-[3-(2-propenyloxy)phenyl]methyl-quinoline-6-carboxamide (hereinafter referred to as the invented compound (9)).

### The Invented Compound (9)

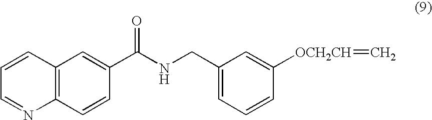

¹H-NMR (CDCl₃) δ: 4.55 (2H, d, J=5.4 Hz), 4.69 (2H, d, J=5.6 Hz), 5.28 (1H, dd, J=10.5, 1.2 Hz), 5.41 (1H, dd, J=17.3, 1.5 Hz), 6.00-6.10 (1H, m), 6.56 (1H, br s), 6.86-6.89 (1H, m), 6.96-6.99 (2H, m), 7.27-7.31 (1H, m), 7.47 (1H, dd, J=8.2, 4.3 Hz), 8.07 (1H, dd, J=8.8, 2.0 Hz), 8.16 (1H, d, J=8.8 Hz), 8.24 (1H, d, J=8.0 Hz), 8.33 (1H, d, J=1.5 Hz), 8.99 (1H, dd, J=4.1, 1.7 Hz).

### Preparation Example 10 of the Invented Compound

To a mixture of 0.30 g of N-(3-hydroxyphenyl)methyl-quinoline-6-carboxamide, 52 mg of sodium hydride (55% dispersion in oil), 20 mg of potassium iodide and 5 ml of DMF was added 0.19 g of 4-bromo-1-butene, and the resulting mixture was stirred at 80° C. for 5 hours. Then, the reaction mixture was allowed to cool to about room temperature and water was added thereto, followed by extraction with ethyl acetate. The organic layer was washed with a saturated aqueous sodium chloride solution, dried over magnesium sulfate and then concentrated under reduced pressure. After the concentration, the residue was subjected to silica gel column chromatography to obtain 35 mg of N-[3-(3-butenyloxy)phenyl]methyl-quinoline-6-carboxamide (hereinafter referred to as the invented compound (10)).

### The Invented Compound (10)

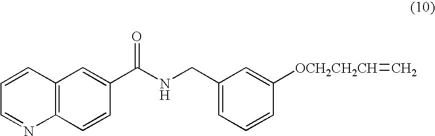

¹H-NMR (CDCl₃) δ: 8.99 (1H, d, J=2.4 Hz), 8.33 (1H, s), 8.24 (1H, d, J=8.0 Hz), 8.16 (1H, d, J=8.8 Hz), 8.07 (1H, dd, J=8.8, 1.7 Hz), 7.48 (1H, dd, J=8.2, 4.3 Hz), 7.30 (1H, d, J=7.8 Hz), 6.98-6.94 (2H, m), 6.87-6.85 (1H, m), 6.52 (1H, s), 5.95-5.85 (1H, m), 5.19-5.09 (2H, m), 4.68 (2H, d, J=5.6 Hz), 4.03 (2H, t, J=6.7 Hz), 2.57-2.52 (2H, m).

### Preparation Example 11 of the Invented Compound

To a mixture of 0.50 g of N-(3-hydroxyphenyl)methyl-quinoline-6-carboxamide, 0.32 g of 5-bromo-1-pentene and 5 ml of DMF was added 0.88 g of cesium carbonate, and the resulting mixture was stirred at room temperature for 12 hours. Then, water was added to the reaction mixture and the solid precipitated was collected by filtration and washed with water and then hexane to obtain 0.57 g of N-[3-(4-pentenyloxy)phenyl]methyl-quinoline-6-carboxamide (hereinafter referred to as the invented compound (11)).

### The Invented Compound (11)

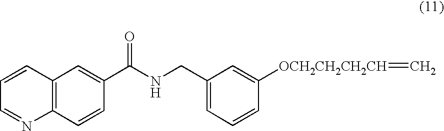

¹H-NMR (DMSO-D₆) δ: 9.26 (1H, t, J=5.9 Hz), 8.99 (1H, d, J=3.7 Hz), 8.56 (1H, s), 8.48 (1H, d, J=8.0 Hz), 8.22 (1H, d, J=8.8 Hz), 8.09 (1H, d, J=9.0 Hz), 7.61 (1H, dd, J=8.3, 4.1 Hz), 7.24 (1H, t, J=7.8 Hz), 6.94-6.92 (2H, m), 6.82 (1H, d, J=9.3 Hz), 5.89-5.79 (1H, m), 5.05-4.95 (2H, m), 4.52 (2H, d, J=5.9 Hz), 3.95 (2H, t, J=6.2 Hz), 2.19-2.14 (2H, m), 1.82-1.75 (2H, m).

### Preparation Example 12 of the Invented Compound

To a mixture of 0.50 g of N-(3-hydroxyphenyl)methyl-quinoline-6-carboxamide, 0.35 g of 6-bromo-1-hexene and 5 ml of DMF was added 0.88 g of cesium carbonate, and the resulting mixture was stirred at room temperature for 12 hours. Then, water was added to the reaction mixture and the solid precipitated was collected by filtration and washed with water and then hexane to obtain 0.57 g of N-[3-(5-hexenyloxy)phenyl]methyl-quinoline-6-carboxamide (hereinafter referred to as the invented compound (12)).

### The Invented Compound (12)

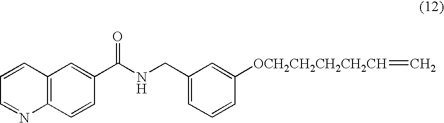

¹H-NMR (DMSO-D₆) δ: 9.26 (1H, t, J=5.7 Hz), 8.99 (1H, dd, J=4.1, 1.5 Hz), 8.56 (1H, d, J=1.7 Hz), 8.48 (1H, d, J=8.0 Hz), 8.22 (1H, dd, J=8.8, 2.0 Hz), 8.09 (1H, d, J=8.8 Hz), 7.61 (1H, dd, J=8.2, 4.3 Hz), 7.24 (1H, t, J=8.0 Hz), 6.93-6.91 (2H, m), 6.83-6.80 (1H, m), 5.85-5.75 (1H, m), 5.01 (1H, dd, J=17.2, 1.6 Hz), 4.94 (1H, dd, J=10.1, 1.1 Hz), 4.51 (2H, d, J=5.9 Hz), 3.95 (2H, t, J=6.5 Hz), 2.09-2.04 (2H, m), 1.73-1.67 (2H, m), 1.52-1.45 (2H, m).

### Preparation Example 13 of the Invented Compound

To a mixture of 0.30 g of N-(3-hydroxyphenyl)methyl-quinoline-6-carboxamide, 0.23 g of 7-bromo-1-heptene and 4 ml of DMF was added 0.53 g of cesium carbonate, and the resulting mixture was stirred at room temperature for 8 hours. Water was added to the reaction mixture and the solid precipitated was collected by filtration and washed with water and then hexane to obtain 0.35 g of N-[3-(6-heptenyloxy)phenyl]methyl-quinoline-6-carboxamide (hereinafter referred to as the invented compound (13)).

### The Invented Compound (13)

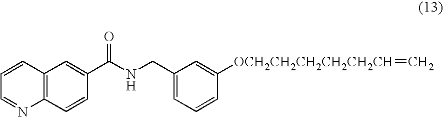

¹H-NMR (DMSO-D₆) δ: 9.26 (1H, t, J=5.9 Hz), 8.99 (1H, dd, J=4.3, 1.6 Hz), 8.56 (1H, d, J=1.7 Hz), 8.48 (1H, d, J=7.6 Hz), 8.22 (1H, dd, J=8.8, 2.0 Hz), 8.09 (1H, d, J=8.8 Hz), 7.62 (1H, dd, J=8.3, 4.1 Hz), 7.24 (1H, t, J=8.2 Hz), 6.93-6.91 (2H, m), 6.82-6.80 (1H, m), 5.83-5.73 (1H, m), 4.99 (1H, dd, J=17.3, 1.5 Hz), 4.92 (1H, dt, J=10.2, 1.0 Hz), 4.51 (2H, d, J=5.9 Hz), 3.94 (2H, t, J=6.5 Hz), 2.04-1.99 (2H, m), 1.73-1.66 (2H, m), 1.41-1.37 (4H, m).

### Preparation Example 14 of the Invented Compound

To a mixture of 0.30 g of N-(3-hydroxyphenyl)methyl-quinoline-6-carboxamide, 0.25 g of 8-bromo-1-octene and 4 ml of DMF was added 0.33 g of cesium carbonate, and the resulting mixture was stirred at room temperature for 8 hours. Water was added to the reaction mixture and the solid precipitated was collected by filtration and washed with water and then hexane to obtain 0.38 g of N-[3-(7-octenyloxy)phenyl]methyl-quinoline-6-carboxamide (hereinafter referred to as the invented compound (14)).

### The Invented Compound (14)

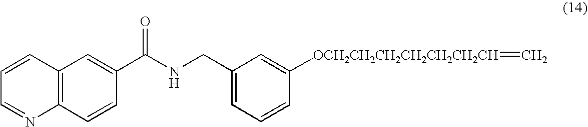

¹H-NMR (DMSO-D₆) δ: 9.26 (1H, t, J=5.9 Hz), 8.99 (1H, dd, J=4.4, 1.7 Hz), 8.56 (1H, d, J=2.0 Hz), 8.48 (1H, d, J=7.6 Hz), 8.22 (1H, dd, J=8.8, 2.0 Hz), 8.09 (1H, d, J=8.8 Hz), 7.62 (1H, dd, J=8.3, 4.1 Hz), 7.24 (1H, t, J=8.0 Hz), 6.93-6.91 (2H, m), 6.82-6.80 (1H, m), 5.82-5.72 (1H, m), 5.01-4.95 (1H, m), 4.94-4.90 (1H, m), 4.51 (2H, d, J=5.9 Hz), 3.93 (2H, t, J=6.5 Hz), 2.02-1.97 (2H, m), 1.72-1.65 (2H, m), 1.43-1.27 (6H, m).

### Preparation Example 15 of the Invented Compound

To a mixture of 0.50 g of N-(3-hydroxyphenyl)methyl-quinoline-6-carboxamide, 0.37 g of potassium carbonate and 4 ml of DMF was added 0.36 g of 4-bromo-2-butene, and the resulting mixture was stirred at room temperature for 8 hours. Then, water was added to the reaction mixture and the solid precipitated was collected by filtration and washed with water and then hexane to obtain 0.33 g of N-[3-(2-butenyloxy)phenyl]methyl-quinoline-6-carboxamide (hereinafter referred to as the invented compound (15)).

### The Invented Compound (15)

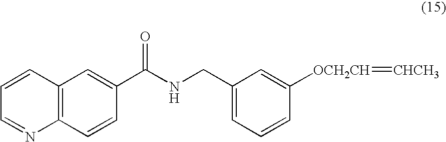

¹H-NMR (CDCl₃) δ: 1.72-1.75 (3.0H, m), 4.46 (1.6H, d, J=6.1 Hz), 4.60 (0.4H, d, J=6.1 Hz), 4.68 (2.0H, d, J=5.6 Hz), 5.68-5.75 (1.2H, m), 5.82-5.90 (0.8H, m), 6.56 (1.0H, s), 6.85-6.89 (1.0H, m), 6.95-6.98 (2.0H, m), 7.26-7.31 (1.0H, m), 7.47 (1.0H, dd, J=8.3, 4.1 Hz), 8.07 (1.0H, dd, J=8.8, 2.0 Hz), 8.15 (1.0H, d, J=8.8 Hz), 8.22-8.25 (1.0H, m), 8.33 (1.0H, d, J=2.0 Hz), 8.99 (1.0H, dd, J=4.4, 1.7 Hz).

### Preparation Example 16 of the Invented Compound

To a mixture of 0.30 g of N-(3-hydroxyphenyl)methyl-quinoline-6-carboxamide, 0.22 g of potassium carbonate and 5 ml of DMF was added 0.19 g of 1-bromo-3-pentene, and the resulting mixture was stirred at room temperature for 8 hours. Then, water was added to the reaction mixture and the solid precipitated was collected by filtration and washed with water and then hexane to obtain 0.24 g of N-[3-(2-pentenyloxy)phenyl]methyl-quinoline-6-carboxamide (hereinafter referred to as the invented compound (16)).

### The Invented Compound (16)

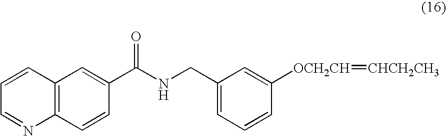

¹H-NMR (CDCl₃) δ: 0.99-1.03 (3.0H, m), 2.06-2.18 (2.0H, m), 4.48 (1.2H, d, J=6.1 Hz), 4.59 (0.8H, d, J=5.4 Hz), 4.69 (2.0H, d, J=5.6 Hz), 5.61-5.72 (1.4H, m), 5.86-5.93 (0.6H, m), 6.53 (1.0H, s), 6.86-6.88 (1.0H, m), 6.95-6.99 (2.0H, m), 7.26-7.31 (1.0H, m), 7.48 (1.0H, dd, J=8.3, 4.1 Hz), 8.07 (1.0H, dd, J=8.8, 2.0 Hz), 8.16 (1.0H, d, J=8.8 Hz), 8.23-8.25 (1.0H, m), 8.33 (1.0H, d, J=2.0 Hz), 8.99 (1.0H, dd, J=4.1, 1.7 Hz).

### Preparation Example 17 of the Invented Compound

To a mixture of 0.17 g of N-(2-fluoro-3-hydroxyphenyl)methyl-quinoline-6-carboxamide, 0.12 g of 3-bromo-1-propyne and 3 ml of DMF was added 0.25 g of cesium carbonate, and the resulting mixture was stirred at room temperature for 12 hours. Water was added to the reaction mixture and the solid precipitated was collected by filtration and washed successively with aqueous sodium hydroxide solution, water and hexane to obtain 0.12 g of N-[2-fluoro-3-(2-propynyloxy)phenyl]methyl-quinoline-6-carboxamide (hereinafter referred to as the invented compound (17)).

### The Invented Compound (17)

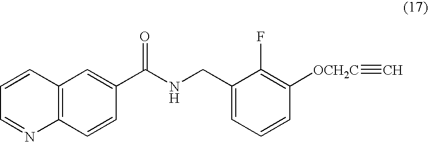

¹H-NMR (CDCl₃) δ: 2.54-2.56 (1H, m), 4.76-4.80 (4H, m), 6.69 (1H, br s), 7.07-7.12 (3H, m), 7.46-7.49 (1H, m), 8.05-8.07 (1H, m), 8.15 (1H, d, J=8.5 Hz), 8.24 (1H, d, J=8.0 Hz), 8.31 (1H, s), 8.99 (1H, s).

### Preparation Example 18 of the Invented Compound

To a mixture of 0.30 g of N-(2-fluoro-3-hydroxyphenyl)methyl-quinoline-6-carboxamide, 0.16 g of 5-chloro-1-pentyne and 5 ml of DMF was added 0.43 g of cesium carbonate, and the resulting mixture was stirred at room temperature for 12 hours. Then, water was added to the reaction mixture and the solid precipitated was collected by filtration and washed with water and then hexane to obtain 0.17 g of N-[2-fluoro-3-(4-pentynyloxy)phenyl]methyl-quinoline-6-carboxamide (hereinafter referred to as the invented compound (18)).

### The Invented Compound (18)

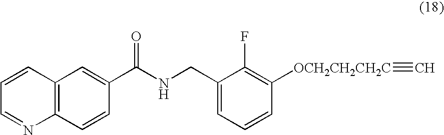

¹H-NMR (CDCl₃) δ: 8.99 (1H, d, J=2.4 Hz), 8.32 (1H, s), 8.24 (1H, d, J=8.0 Hz), 8.15 (1H, d, J=9.0 Hz), 8.06 (1H, d, J=9.0 Hz), 7.47 (1H, dd, J=8.4, 4.0 Hz), 7.06-7.04 (2H, m), 6.95-6.93 (1H, m), 6.67 (1H, s), 4.76 (2H, d, J=5.6 Hz), 4.15 (2H, t, J=6.1 Hz), 2.47-2.43 (2H, m), 2.08-2.02 (2H, m), 1.98 (1H, s).

### Preparation Example 19 of the Invented Compound

To a mixture of 0.30 g of N-(2-fluoro-3-hydroxyphenyl)methyl-quinoline-6-carboxamide, 54 mg of sodium hydride (55% dispersion in oil) and 5 ml of DMF was added 0.15 g of 6-chloro-1-hexyne, and the resulting mixture was stirred at 80° C. for 5 hours and then at 100° C. for 5 hours. Thereafter, the reaction mixture was allowed to cool to about room temperature and water was added thereto, followed by extraction with ethyl acetate. The organic layer was washed with saturated aqueous sodium chloride solution, dried over magnesium sulfate and then concentrated under reduced pressure. After the concentration, the residue was subjected to silica gel column chromatography to obtain 0.26 g of N-[2-fluoro-3-(5-hexynyloxy)phenyl]methyl-quinoline-6-carboxamide (hereinafter referred to as the invented compound (19)).

### The Invented Compound (19)

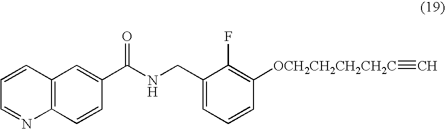

¹H-NMR (CDCl₃) δ: 8.99 (1H, dd, J=4.4, 1.7 Hz), 8.31 (1H, d, J=2.0 Hz), 8.24 (1H, d, J=8.3 Hz), 8.15 (1H, d, J=8.8 Hz), 8.06 (1H, dd, J=8.8, 2.0 Hz), 7.47 (1H, dd, J=8.3, 4.1 Hz), 7.07-7.01 (2H, m), 6.95-6.91 (1H, m), 6.65 (1H, s), 4.76 (2H, d, J=6.1 Hz), 4.07 (2H, t, J=6.3 Hz), 2.30 (2H, td, J=7.0, 2.6 Hz), 2.00-1.93 (3H, m), 1.79-1.72 (2H, m).

### Preparation Example 20 of the Invented Compound

To a mixture of 0.30 g of N-(2-fluoro-3-hydroxyphenyl)methyl-quinoline-6-carboxamide, 0.30 g of potassium carbonate and 5 ml of DMF was added 0.17 g of 4-bromo-2-butyne, and the resulting mixture was stirred at room temperature for 8 hours. Then, water was added to the reaction mixture and the solid precipitated was collected by filtration and washed with water and then hexane to obtain 0.33 g of N-[3-(2-butynyloxy)-2-fluorophenyl]methyl-quinoline-6-carboxamide (hereinafter referred to as the invented compound (20)).

### The Invented Compound (20)

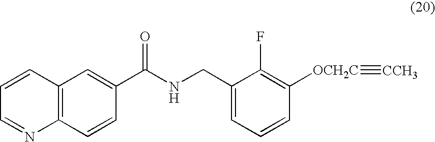

¹H-NMR (CDCl₃) δ: 1.85 (3H, s), 4.73-4.76 (4H, m), 6.77 (1H, s), 7.05-7.07 (3H, m), 7.45-7.48 (1H, m), 8.05-8.07 (1H, m), 8.14 (1H, d, J=8.7 Hz), 8.23 (1H, d, J=7.0 Hz), 8.30-8.31 (1H, m), 8.97-8.98 (1H, m).

### Preparation Example 21 of the Invented Compound

To a mixture of 0.30 g of N-(2-fluoro-3-hydroxyphenyl)methyl-quinoline-6-carboxamide, 0.18 g of 1-bromo-2-pentyne and 4 ml of DMF was added 0.50 g of cesium carbonate, and the resulting mixture was stirred at room temperature for 12 hours. Water was added to the reaction mixture and the solid precipitated was collected by filtration and washed with water and then hexane to obtain 0.26 g of N-[2-fluoro-3-(2-pentynyloxy)phenyl]methyl-quinoline-6-carboxamide (hereinafter referred to as the invented compound (21)).

### The Invented Compound (21)

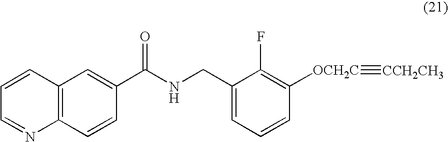

¹H-NMR (CDCl₃) δ: 8.99 (1H, dd, J=4.1, 1.7 Hz), 8.31 (1H, d, J=2.0 Hz), 8.24 (1H, d, J=7.3 Hz), 8.15 (1H, d, J=8.8 Hz), 8.06 (1H, dd, J=8.9, 2.1 Hz), 7.47 (1H, dd, J=8.3, 4.1 Hz), 7.08-7.07 (3H, m), 6.65 (1H, s), 4.78-4.75 (4H, m), 2.25-2.19 (2H, m), 1.12 (3H, q, J=5.9 Hz).

### Preparation Example 22 of the Invented Compound

To a mixture of 0.19 g of N-(3-hydroxyphenyl)methyl-quinoline-6-carboxamide, 0.12 g of 3-bromo-1-propyne and 3 ml of DMF was added 0.25 g of cesium carbonate, and the resulting mixture was stirred at room temperature for 12 hours. Water was added to the reaction mixture and the solid precipitated was collected by filtration and washed successively with aqueous sodium hydroxide solution, water and hexane to obtain 0.16 g of N-[3-(2-propynyloxy)phenyl]methyl-quinoline-6-carboxamide (hereinafter referred to as the invented compound (22)).

### The Invented Compound (22)

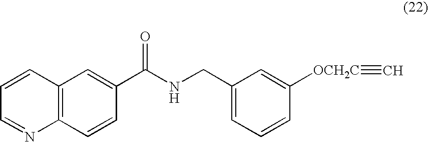

¹H-NMR (CDCl₃) δ: 2.50 (1H, t, J=2.3 Hz), 4.70-4.71 (4H, m), 6.57 (1H, br s), 6.93-6.95 (1H, m), 7.02-7.05 (2H, m), 7.30-7.34 (1H, m), 7.48 (1H, dd, J=8.3, 4.1 Hz), 8.05-8.08 (1H, m), 8.16 (1H, d, J=9.0 Hz), 8.24 (1H, d, J=8.3 Hz), 8.34 (1H, s), 8.99 (1H, d, J=4.1 Hz).

### Preparation Example 23 of the Invented Compound

To a mixture of 0.30 g of N-(3-hydroxyphenyl)methyl-quinoline-6-carboxamide, 54 mg of sodium hydride (55% dispersion in oil), 20 mg of potassium iodide and 5 ml of DMF was added 0.14 g of 5-chloro-1-pentyne, and the resulting mixture was stirred at 80° C. for 5 hours. Then, the reaction mixture was allowed to cool to about room temperature and water was added thereto, followed by extraction with ethyl acetate. The organic layer was washed with a saturated aqueous sodium chloride solution, dried over magnesium sulfate and then concentrated under reduced pressure. After the concentration, the residue was subjected to silica gel column chromatography to obtain 0.34 g of N-[3-(4-pentynyloxy)phenyl]methyl-quinoline-6-carboxamide (hereinafter referred to as the invented compound (23)).

### The Invented Compound (23)

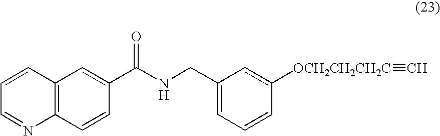

¹H-NMR (CDCl₃) δ: 8.99 (1H, dd, J=4.1, 1.7 Hz), 8.33 (1H, d, J=2.0 Hz), 8.24 (1H, d, J=8.3 Hz), 8.16 (1H, d, J=9.0 Hz), 8.07 (1H, dd, J=8.8, 2.0 Hz), 7.47 (1H, dd, J=8.3, 4.1 Hz), 7.29 (1H, t, J=7.9 Hz), 6.99-6.94 (2H, m), 6.86 (1H, dd, J=8.0, 2.0 Hz), 6.55 (1H, s), 4.69 (2H, d, J=5.6 Hz), 4.08 (2H, t, J=6.0 Hz), 2.41 (2H, td, J=7.0, 2.6 Hz), 2.03-1.98 (2H, m), 1.96 (1H, t, J=2.7 Hz).

### Preparation Example 24 of the Invented Compound

To a mixture of 0.30 g of N-(3-hydroxyphenyl)methyl-quinoline-6-carboxamide, 52 mg of sodium hydride (55% dispersion in oil) and 5 ml of DMF was added 0.15 g of 6-chloro-1-hexyne, and the resulting mixture was stirred at 80° C. for 5 hours and then at 100° C. for 5 hours. Thereafter, the reaction mixture was allowed to cool to about room temperature and water was added thereto, followed by extraction with ethyl acetate. The organic layer was washed with saturated aqueous sodium chloride solution, dried over magnesium sulfate and then concentrated under reduced pressure. After the concentration, the residue was subjected to silica gel column chromatography to obtain 0.26 g of N-[3-(5-hexynyloxy)phenyl]methyl-quinoline-6-carboxamide (hereinafter referred to as the invented compound (24)).

### The Invented Compound (24)

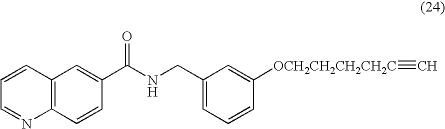

¹H-NMR (CDCl₃) δ: 8.94 (1H, dd, J=4.3, 1.3 Hz), 8.42 (1H, s), 8.37 (1H, d, J=8.3 Hz), 8.27 (1H, d, J=8.8 Hz), 8.16 (1H, dd, J=8.8, 1.5 Hz), 7.55 (1H, dd, J=8.3, 4.4 Hz), 7.29-7.25 (1H, m), 6.99-6.95 (3H, m), 6.84-6.82 (1H, m), 4.68 (2H, d, J=5.6 Hz), 3.98 (2H, t, J=6.2 Hz), 2.27 (2H, td, J=7.0, 2.5 Hz), 1.96 (1H, t, J=2.6 Hz), 1.94-1.87 (2H, m), 1.75-1.67 (2H, m).

### Preparation Example 25 of the Invented Compound

To a mixture of 0.30 g of N-(3-hydroxyphenyl)methyl-quinoline-6-carboxamide, 0.30 g of potassium carbonate and 5 ml of DMF was added 0.16 g of 4-bromo-2-butyne, and the resulting mixture was stirred at room temperature for 8 hours. Then, water was added to the reaction mixture and the solid precipitated was collected by filtration and washed with water and then hexane to obtain 0.28 g of N-[3-(2-butynyloxy)phenyl]methyl-quinoline-6-carboxamide (hereinafter referred to as the invented compound (25)).

### The Invented Compound (25)

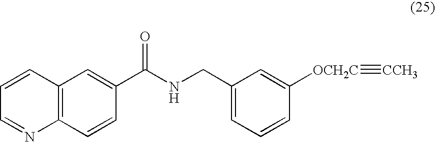

¹H-NMR (CDCl₃) δ: 1.82 (3H, t, J=2.3 Hz), 4.65 (2H, q, J=2.2 Hz), 4.69 (2H, d, J=5.6 Hz), 6.65 (1H, s), 6.90-6.93 (1H, m), 6.99-7.02 (2H, m), 7.30 (1H, t, J=8.2 Hz), 7.47 (1H, dd, J=8.3, 4.2 Hz), 8.07 (1H, dd, J=8.7, 1.9 Hz), 8.14 (1H, d, J=8.7 Hz), 8.23 (1H, d, J=8.2 Hz), 8.33 (1H, d, J=1.9 Hz), 8.98 (1H, dd, J=4.2, 1.6 Hz).

### Preparation Example 26 of the Invented Compound

To a mixture of 0.30 g of N-(3-hydroxyphenyl)methyl-quinoline-6-carboxamide, 0.19 g of 1-bromo-2-pentyne and 4 ml of DMF was added 0.53 g of cesium carbonate, and the resulting mixture was stirred at room temperature for 12 hours. Water was added to the reaction mixture and the solid precipitated was collected by filtration and washed with water and then hexane to obtain 0.33 g of N-[3-(2-pentynyloxy)phenyl]methyl-quinoline-6-carboxamide (hereinafter referred to as the invented compound (26)).

### The Invented Compound (26)

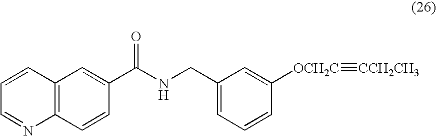

¹H-NMR (CDCl₃) δ: 8.98 (1H, dd, J=4.1, 1.7 Hz), 8.33 (1H, d, J=1.7 Hz), 8.23 (1H, d, J=7.8 Hz), 8.15 (1H, d, J=8.8 Hz), 8.07 (1H, dd, J=8.8, 2.0 Hz), 7.47 (1H, dd, J=8.3, 4.1 Hz), 7.30 (1H, t, J=8.2 Hz), 7.01-7.00 (2H, m), 6.94-6.91 (1H, m), 6.65 (1H, s), 4.70-4.67 (4H, m), 2.23-2.17 (2H, m), 1.10 (3H, t, J=7.6 Hz).

### Preparation Example 27 of the Invented Compound

To a mixture of 0.50 g of N-(2-fluoro-3-hydroxyphenyl)methyl-quinoline-6-carboxamide, 0.38 g of potassium carbonate and 10 ml of acetonitrile was added 2.5 g of 3-butyne p-toluenesulfonate, and the resulting mixture was stirred for 36 hours under reflux conditions. Then, water was added to the reaction mixture, followed by extraction with ethyl acetate. The organic layer was washed with 3% aqueous sodium hydroxide solution, water and saturated aqueous sodium chloride solution, dried over magnesium sulfate and then concentrated under reduced pressure. The residue was subjected to silica gel column chromatography to obtain 0.24 g of N-[3-(3-butynyloxy)-2-fluorophenyl]methyl-quinoline-6-carboxamide (hereinafter referred to as the invented compound (27)).

### The Invented Compound (27)

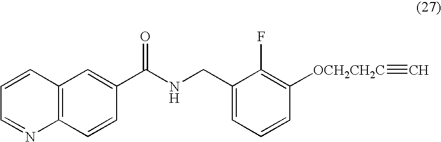

¹H-NMR (CDCl₃) δ: 2.05 (1H, t, J=2.9 Hz), 2.73 (2H, td, J=7.1, 2.7 Hz), 4.18 (2H, t, J=7.1 Hz), 4.76 (2H, d, J=5.8 Hz), 6.65 (1H, s), 6.93-6.98 (1H, m), 7.04-7.08 (2H, m), 7.48 (1H, dd, J=8.1, 4.2 Hz), 8.06 (1H, dd, J=8.8, 2.1 Hz), 8.16 (1H, d, J=8.7 Hz), 8.23-8.26 (1H, m), 8.31 (1H, d, J=1.9 Hz), 8.99 (1H, dd, J=4.3, 1.4 Hz).

### Preparation Example 28 of the Invented Compound

To a mixture of 0.50 g of N-(3-hydroxyphenyl)methyl-quinoline-6-carboxamide, 0.37 g of potassium carbonate and 5 ml of acetonitrile was added 2.4 g of 3-butyne p-toluenesulfonate, and the resulting mixture was stirred for 36 hours under reflux conditions. Then, water was added to the reaction mixture, followed by extraction with ethyl acetate. The organic layer was washed with 3% aqueous sodium hydroxide solution, water and saturated aqueous sodium chloride solution, dried over magnesium sulfate and then concentrated under reduced pressure. The residue was subjected to silica gel column chromatography to obtain 0.10 g of N-[3-(3-butynyloxy)phenyl]methyl-quinoline-6-carboxamide (hereinafter referred to as the invented compound (28)).

### The Invented Compound (28)

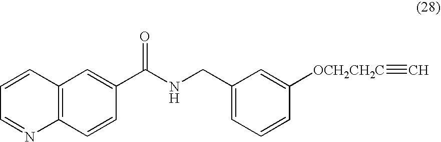

¹H-NMR (CDCl₃) δ: 2.03 (1H, t, J=2.7 Hz), 2.68 (2H, td, J=6.9, 2.7 Hz), 4.11 (2H, t, J=7.0 Hz), 4.69 (2H, d, J=5.6 Hz), 6.54 (1H, s), 6.87 (1H, dd, J=8.2, 1.9 Hz), 6.94-7.01 (2H, m), 7.30 (1H, t, J=7.8 Hz), 7.48 (1H, dd, J=8.3, 4.2 Hz), 8.07 (1H, dd, J=8.8, 2.1 Hz), 8.16 (1H, d, J=8.7 Hz), 8.24 (1H, d, J=8.0 Hz), 8.33 (1H, d, J=1.9 Hz), 8.99 (1H, dd, J=4.1, 1.7 Hz).

### Preparation Example 29 of the Invented Compound

To a mixture of 0.15 g of 6-quinolinecarboxylic acid, 0.20 g of [2-fluoro-3-(4-pentynyloxy)phenyl]methylamine hydrochloride, 0.30 ml of triethylamine and 5 ml of DMF was added 0.44 g of BOP reagent, and the resulting mixture was stirred at room temperature for 3 hours. Then, water was added to the reaction mixture, followed by extraction with ethyl acetate. The organic layer was washed with 3% aqueous sodium hydroxide solution, water and saturated aqueous sodium chloride solution, dried over magnesium sulfate and then concentrated under reduced pressure. The residue was filtered together with small amounts of ethyl acetate and hexane. The resultant solid was washed with hexane and dried to obtain 0.16 g of the invented compound (2).

### Synthesis Example of the Present Amine Compound

At room temperature, 17 g of 1,1-dimethylethyl (2-fluoro-3-(4-pentynyloxy)phenyl)methylcarbamate, 17 ml of trifluoroacetic acid and 50 ml of chloroform were mixed for 5 hours. The resulting reaction mixture was concentrated under reduced pressure and a mixture of 100 ml or ethanol and 6 ml of acetyl chloride was added to the residue, after which the resultant mixture was concentrated under reduced pressure to obtain 8.7 g of [2-fluoro-3-(4-pentynyloxy)phenyl]methylamine hydrochloride.

### The Present Amine Compound Produced

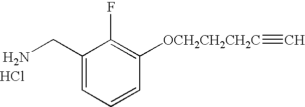

¹H-NMR (DMSO-D6) δ: 1.88-1.95 (2H, m), 2.34 (2H, td, J=7.0, 2.6 Hz), 2.84 (1H, dd, J=2.7, 1.9 Hz), 4.01-4.04 (2H, m), 4.13 (2H, t, J=6.0 Hz), 7.12-7.24 (3H, m), 8.51 (3H, s).

### Preparation Example 1 of an Intermediate

To 4 g of 2-fluoro-3-methoxybenzylamine hydrochloride was added 15 ml of 48% hydrobromic acid, and the resulting mixture was heated under reflux for 5 hours. The reaction mixture was allowed to cool to about room temperature and concentrated under reduced pressure to obtain 4.0 g of 2-fluoro-3-hydroxybenzylamine hydrobromide.

### 2-Fluoro-3-hydroxybenzylamine hydrobromide

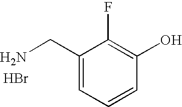

¹H-NMR (DMSO-d₆) δ: 4.04 (2H, q, J=5.5 Hz), 6.90-6.94 (1H, m), 6.97-7.06 (2H, m), 8.23 (3H, br s), 10.05 (1H, br s).

### Preparation Example 2 of an Intermediate

To a mixture of 2.59 g of 6-quinolinecarboxylic acid, 3.33 g of 2-fluoro-3-hydroxybenzylamine hydrobromide and 20 ml of pyridine was added 3.33 g of WSC, and the resulting mixture was stirred at room temperature for 12 hours. Water was added to the reaction mixture and the solid precipitated was collected by filtration, washed successively with saturated aqueous sodium hydrogencarbonate solution, water and hexane, and then dried to obtain 2.6 g of N-(2-fluoro-3-hydroxyphenyl)methyl-quinoline-6-carboxamide.

### N-(2-Fluoro-3-hydroxyphenyl)methyl-quinoline-6-carboxamide

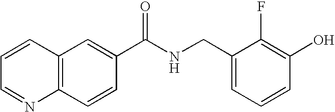

¹H-NMR (DMSO-d₆) δ: 1.70 (1H, s), 4.55-4.57 (2H, m), 6.75-6.93 (3H, m), 7.60-7.64 (1H, m), 8.10 (1H, d, J=8.0 Hz), 8.22 (1H, d, J=8.0 Hz), 8.48 (1H, d, J=7.3 Hz), 8.57 (1H, s), 8.99 (1H, s), 9.25 (1H, s).

### Preparation Example 3 of an Intermediate

A mixture of 20 g of 3-hydroxybenzaldehyde, 200 ml of 2-propanol, 16 g of pyridine and 15 g of hydroxylamine hydrochloride was stirred at room temperature for 3 hours. The reaction mixture was concentrated under reduced pressure and water was added thereto, followed by extraction with ethyl acetate. The organic layer was washed successively with 5% hydrochloric acid, water and saturated aqueous sodium chloride solution, dried over magnesium sulfate, and then concentrated under reduced pressure to obtain 26 g of 3-hydroxybenzaldoxime.

A mixture of 25 g of the 3-hydroxybenzaldoxime obtained, 1.3 g of 5% palladium-carbon and 300 ml of ethanol was stirred at room temperature in the presence of hydrogen gas at atmospheric pressure. The reaction mixture which had ceased to absorb hydrogen gas was filtered through Celite®, and the filtrate was concentrated under reduced pressure. The resultant residue, 200 ml of acetonitrile and 28 g of concentrated hydrochloric acid were mixed at 0° C., and the solid formed was collected by filtration, washed with acetonitrile and then dried under reduced pressure to obtain 20 g of 3-hydroxybenzylamine hydrochloride.

### 3-Hydroxybenzylamine hydrochloride

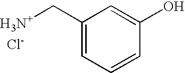

¹H-NMR (DMSO-d₆) δ: 3.89 (2H, q, J=4.0 Hz), 6.79-6.81 (1H, m), 6.89-6.91 (2H, m), 7.18 (1H, t, J=7.9 Hz), 8.51 (3H, br s), 9.72 (1H, br s).

### Preparation Example 4 of an Intermediate

A mixture of 9.4 g of 3-hydroxybenzylamine hydrochloride, 9.3 g of 6-quinolinecarboxylic acid, 12.3 g of WSC, 22 ml of pyridine and 100 ml of DMF was stirred at room temperature for 3 hours. Saturated aqueous ammonium chloride solution was added to the reaction mixture, followed by extraction with ethyl acetate. The organic layer was washed with saturated aqueous sodium chloride solution, dried over magnesium sulfate, and then concentrated under reduced pressure. Toluene was added to the residue, and the crystals precipitated were collected by filtration, washed with toluene and hexane, and then dried under reduced pressure to obtain 9.8 g of N-(3-hydroxyphenyl)methyl-quinoline-6-carboxamide.

### N-(3-Hydroxyphenyl)methyl-quinoline-6-carboxamide

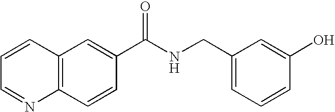

¹H-NMR (DMSO-d₆) δ: 4.47 (2H, d, J=6.0 Hz), 6.63-6.65 (1H, m), 6.77-6.79 (2H, m), 7.13 (1H, t, J=7.3 Hz), 7.62 (1H, dd, J=8.0, 4.0 Hz), 8.10 (1H, d, J=8.4 Hz), 8.23 (1H, dd, J=8.8, 1.7 Hz), 8.48 (1H, d, J=7.6 Hz), 8.57 (1H, d, J=1.2 Hz), 8.99-9.00 (1H, m), 9.24 (1H, t, J=6.0 Hz), 9.34 (1H, s).

### Preparation Example 5 of an Intermediate

At room temperature, 26 g of 2-fluoro-3-hydroxybenzylamine hydrobromide, 49 g of di-tert-butyl dicarbonate, 64 ml of triethylamine, about 0.1 g of 4-dimethylaminopyridine and 300 ml of tetrahydrofuran were mixed for 6 hours. Then, water was added to the reaction mixture, followed by extraction with ethyl acetate. The organic layer was separated, washed successively with 5% hydrochloric acid, water, saturated aqueous sodium hydrogencarbonate solution and saturated aqueous sodium chloride solution, dried over magnesium sulfate, and then concentrated under reduced pressure to obtain about 20 g of a crude product of 1,1-dimethylethyl [2-fluoro-3-(4-hydroxy)phenyl]methylcarbamate.

A mixture of about 20 g of the crude product of 1,1-dimethylethyl [2-fluoro-3-(4-hydroxy)phenyl]methylcarbamate, 11 g of 5-chloro-1-pentyne, 44 g of cesium carbonate and 200 ml of DMF was stirred at 80° C. for 4 hours and then at 100° C. for 2 hours. Thereafter, the reaction mixture was allowed to cool to about room temperature and water was added thereto, followed by extraction with ethyl acetate. The organic layer was washed successively with 3% aqueous sodium hydroxide solution, 5% hydrochloric acid, water, saturated aqueous sodium hydrogencarbonate solution and saturated aqueous sodium chloride solution, dried over magnesium sulfate, and then concentrated under reduced pressure. After the concentration, the residue was subjected to silica gel column chromatography to obtain 18 g of 1,1-dimethylethyl [2-fluoro-3-(4-pentynyloxy)phenyl]methylcarbamate.

### 1,1-Dimethylethyl [2-fluoro-3-(4-pentynyloxy)phenyl]methylcarbamate

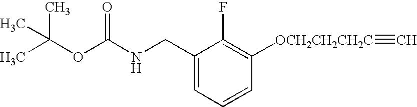

¹H-NMR (CDCl₃) δ: 1.45 (9H, s), 1.97 (1H, t, J=2.7

Hz), 2.00-2.06 (2H, m), 2.43 (2H, td, J=7.0, 2.7 Hz), 4.13 (2H, t, J=6.1 Hz), 4.35-4.37 (2H, m), 4.88 (1H, s), 6.89-6.92 (1H, m), 6.99-7.03 (1H, m), 7.07-7.14 (1H, m).

### Preparation Example 6 of Intermediate

4.5 g of 2-fluoro-3-methoxybenzyl alcohol, 2.9 ml of methansulfonyl chloride and 50 ml of THF were mixed and stirred at 0° C. To the mixture was dropped 6.0 ml of triethylamine, followed by stirring at 0° C. for 30 minutes and then at room temperature for 2 hours. Thereafter, ethyl acetate was added to the reaction mixture left to stand for cooling to about room temperature, and then the mixture was filtered by passing through Celite®. Water was added to the filtrate, followed by extracting with ethyl acetate. The organic layer was washed successively with water and saturated aqueous sodium chloride solution and dried over magnesium sulfate, and then concentrated under reduced pressure to obtain 6.9 g of (2-fluoro-3-methoxyphenyl)methyl-methanesulfonate.

### (2-Fluoro-3-methoxyphenyl)methyl-methanesulfonate

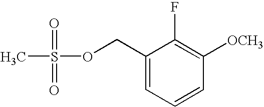

¹H-NMR (CDCl₃) δ: 3.00 (3H, s), 3.91 (3H, s), 5.30 (2H, s), 6.96-7.14 (3H, m).

### Preparation Example 7 of Intermediate

6.7 g of (2-fluoro-3-methoxyphenyl)methyl-methanesulfonate, 5.3 g of potassium phthalimide and 60 ml of DMF were mixed and stirred at 70° C. for 4 hours. Then, water was added to the reaction mixture, followed by extracting with ethyl acetate. The organic layer was washed successively with water, diluted hydrochloric acid and saturated aqueous sodium chloride solution and dried over magnesium sulfate, and then concentrated under reduced pressure. The residue was washed with hexane to obtain 5.8 g of N-(2-fluoro-3-methoxyphenyl)methylphthalimide.

### N-(2-Fluoro-3-methoxyphenyl)methylphthalimide

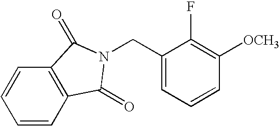

¹H-NMR (CDCl₃) δ: 3.87 (3H, s), 4.94 (2H, s), 6.86-6.90 (2H, m), 6.98-7.02 (1H, m), 7.73 (2H, dd, J=5.4, 3.0 Hz), 7.86 (2H, dd, J=5.4, 3.0 Hz).

### Preparation Example 8 of Intermediate

To a mixture of 7.7 g of N-(2-fluoro-3-methoxyphenyl)methylphthalimide and 30 ml of ethanol was dropped 1.63 g of hydrazine monohydrate, followed by refluxing with heating for 4 hours. Then, the reaction mixture was cooled to room temperature and water was added thereto, and then the mixture was concentrated under reduced pressure. Diluted hydrochloric acid was added to the residue, followed by filtration. To the resulting filtrate was added ethyl acetate, and 15% aqueous sodium hydroxide solution was added thereto until the aqueous layer of the mixed solution became basic, followed by separation. Concentrated hydrochloric acid was added to the resulting organic layer, and then the organic layer was concentrated under reduced pressure to obtain 4.5 g of 2-fluoro-3-methoxybenzylamine hydrochloride.

### 2-Fluoro-3-methoxybenzylamine hydrochloride

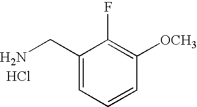

¹H-NMR (DMSO-d₆) δ: 3.85 (3H, s), 4.03 (2H, q, J=5.3 Hz), 7.13-7.23 (3H, m), 8.60 (3H, br s).

### Preparation Example 9 of Intermediate

To a mixed solution of 15.4 g of 2-fluoro-3-methoxybenzaldehyde, 30 ml of THF and 3 ml of water was dropped 24 ml of pyridine, and to the resulting mixture was added 13.8 g of hydroxylamine hydrochloride under ice cooling. The mixture was stirred at room temperature for 30 minutes, and concentrated under reduced pressure until the whole volume reduced to about half. Then, water was added to the residue, followed by extracting with ethyl acetate. The organic layer was washed successively with diluted hydrochloric acid and saturated aqueous sodium chloride solution, and dried over magnesium sulfate, and then concentrated under reduced pressure to obtain 16 g of 2-fluoro-3-methoxybenzaldehyde oxime.

### 2-Fluoro-3-methoxybenzaldehyde oxime

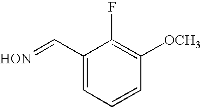

¹H-NMR (CDCl₃) δ: 3.90 (3H, s), 6.96-7.00 (1H, m), 7.03-7.12 (1H, m), 7.30-7.33 (1H, m), 7.64-7.78 (1H, m), 8.39 (1H, s).

### Preparation Example 10 of Intermediate

To a mixed solution of 2.4 g of 10% palladium carbon, 8.3 ml of 10N hydrochloric acid and 200 ml of ethanol was added 12.8 g of 2-fluoro-3-methoxybenzaldehyde oxime, followed by stirring under normal pressure in hydrogen atmosphere. After absorption of hydrogen gas stopped, the reaction mixture was filtered through Celite®. The filtrate was concentrated under reduced pressure to obtain 8.2 g of 2-fluoro-3-methoxybenzylamine hydrochloride.

### Preparation Example 11 of Intermediate

A mixed solution of 10 g of 2-fluoro-3-methoxybenzaldehyde and 80 ml of ethanol was added to a mixed solution of 1.8 g of 10% palladium carbon, 10 ml of water, 6.5 ml of 10N hydrochloric acid and 50 ml of ethanol, and then 5.4 g of hydroxylamine hydrochloride was added thereto. The mixture was stirred at room temperature for 2 hours, followed by stirring under normal pressure in hydrogen atmosphere for 3 hours. The reaction mixture was filtered through Celite®, and the filtrate was concentrated under reduced pressure. To the residue was added 100 ml of water, followed by extracting with chloroform. To the resulting aqueous layer was basified by addition of 15% aqueous sodium hydroxide solution, followed by extracting with chloroform. The resulting organic layer was dried with potassium carbonate and concentrated under reduced pressure to obtain 8.0 g of 2-fluoro-3-methoxybenzylamine.

### 2-Fluoro-3-methoxybenzylamine

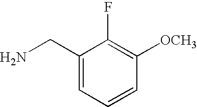

¹H-NMR (CDCl₃) δ: 1.55 (2H, br s), 3.89 (3H, s), 3.90 (2H, br s), 6.86-6.91 (2H, m), 7.02-7.06 (1H, m).

### Formulation Example 1

Fifty parts of one of the invented compounds (1)-(28), 3 parts of calcium ligninsulfonate, 2 parts of magnesium laurylsulfate, and 45 parts of synthetic hydrous silicon oxide are well ground and mixed to obtain a wettable powder.

### Formulation Example 2

Twenty parts of one of the invented compounds (1)-(28) and 1.5 part of sorbitan trioleate are mixed with 28.5 parts of an aqueous solution containing 2 parts of polyvinyl alcohol, and the mixture is pulverized by wet pulverizing method. Then, thereto is added 40 parts of an aqueous solution containing 0.05 part of xanthan gum and 0.1 part of aluminum magnesium silicate, and further added 10 parts of propylene glycol, followed by stirring and mixing to obtain a flowable formulation.

### Formulation Example 3

Two parts of one of the invented compounds (1)-(28), 88 parts of kaolin clay and 10 parts of talc are well ground and mixed to obtain a dust formulation.

### Formulation Example 4

Five parts of one of the invented compounds (1)-(28), 14 parts of polyoxyethylenestyrylphenyl ether, 6 parts of calcium dodecylbenzenesulfonate, and 75 parts of xylene are well mixed to obtain an emulsifiable concentrate.

### Formulation Example 5

Two parts of one of the invented compounds (1)-(28), 1 part of synthetic hydrous silicon oxide, 2 parts of calcium ligninsulfonate, 30 parts of bentonite and 65 parts of kaolin clay are well ground and mixed, then water is added thereto, followed by well kneading and granulation drying to obtain granule formulation.

### Formulation Example 6

Ten parts of one of the invented compounds (1)-(28), 35 parts of white carbon containing 50 parts of polyoxyethylene alkyl ether sulfate ammonium salt and 55 parts of water are mixed, and the mixture is pulverized by wet pulverizing method to obtain a flowable formulation.

Next, usefulness of the invented compounds for controlling plant diseases is shown by test examples.

The area of lesions on the test plants at testing was visually observed, and the control effect was evaluated by comparing the area of lesions on test plants treated with the invented compound with the area of lesions on untreated plants.

As control test, N-[3-(3-methyl-2-butenyloxy)phenyl]methyl-quinoline-6-carboxamide (represented by the following formula (A) and hereinafter referred to as “comparative compound (A)”) which is disclosed in Example E-49 of WO 2005/033079 was further used for test.

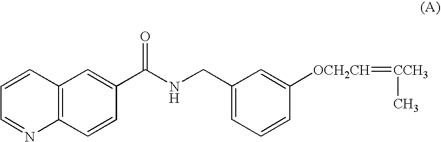

### Test Example 1

### Test for Showing Preventive Effect on Botrytis Disease of Cucumber (Botrytis cinerea)

Sand soil was packed in a plastic pot, and seeds of cucumber (variety: Sagami Hanjiro) were sowed therein and grown for 12 days in a greenhouse. Each of the invented compounds (2), (3), (7), (11), (12), (15), (16), (18), (19), (21), (23), (24) and (27) and the comparative compound (A) was formulated into flowable formulation in accordance with Formulation Example 6, and it was diluted with water to a given concentration (13 ppm) and was sprayed onto the foliage of the cucumber so that a sufficient amount of the compound would be applied to the surface of leaves of the cucumber. After spraying, the plant was air-dried and PDA medium containing spores of Botrytis cinerea was placed on the surface of leaves of the cucumber. After the inoculation, the plant was left at 12° C. for 5 days under high humidity, and then the area of lesions was examined. As a result, the lesion area on the plant treated with the invented compounds (2), (3), (7), (11), (12), (15), (16), (18), (19), (21), (23), (24) and (27) was less than 30% of the lesion area on the untreated plant. The lesion area on the plant treated with the comparative compound (A) was 98% of the untreated plant.

### Test Example 2

### Test for Showing Preventive Effect on Sclerotinia Rot Disease of Cucumber (Sclerotinia sclerotiorum)

Sand soil was packed in a plastic pot, and seeds of cucumber (variety: Sagami Hanjiro) were sowed therein and grown for 12 days in a greenhouse. Each of the invented compounds (1), (3)-(27) and (28) was formulated into flowable formulation in accordance with Formulation Example 6, and it was diluted with water to a given concentration (500 ppm) and was sprayed onto the foliage of the cucumber so that a sufficient amount of the compound would be applied to the surface of leaves of the cucumber. After spraying, the plant was air-dried and PDA medium containing hyphae of Sclerotinia sclerotiorum was placed on the surface of leaves of the cucumber. After the inoculation, the plant was left at 18° C. for 4 days under high humidity, area of lesions was examined. As a result, the lesion area on the plant treated with the invented compounds (1), (3)-(27) and (28) was less than 10% of the lesion area on the untreated plant.

### Test Example 3

### Test for Showing Preventive Effect on Sclerotinia Rot Disease of Cucumber (Sclerotinia sclerotiorum)

Sand soil was packed in a plastic pot, and seeds of cucumber (variety: Sagami Hanjiro) were sowed therein and grown for 12 days in a greenhouse. The invented compound (2) was formulated into flowable formulation in accordance with Formulation Example 6, and it was diluted with water to a given concentration (200 ppm) and was sprayed onto the foliage of the cucumber so that a sufficient amount of the compound would be applied to the surface of leaves of the cucumber. After spraying, the plant was air-dried and PDA medium containing hyphae of Sclerotinia sclerotiorum was placed on the surface of leaves of the cucumber. After the inoculation, the plant was left at 18° C. for 4 days under high humidity, area of lesions was examined. As a result, the lesion area on the plant treated with the invented compound (2) was less than 10% of the lesion area on the untreated plant.

### Test Example 4

Test for Showing Preventive Effect on Rice Blast Disease (Magnaporthe grisea)

Bed soil was packed in a plastic pot, and seeds of rice (variety: Nihonbare) were sowed therein and grown for 12 days in a greenhouse. Each of the invented compounds (1), (7), (9), (15), (17), (18), (20), (25), (27) and (28) and the comparative compound (A) was formulated into flowable formulation in accordance with Formulation Example 6, and it was diluted with water to a given concentration (500 ppm) and was sprayed onto the foliage of the rice so that a sufficient amount of the compound would be applied to the surface of leaves of the rice. After spraying, the plant was air-dried and pots containing leaves infected with blast disease were placed around the sprayed plant. All of the rice were kept under high humidity only in night, and after 5 days from the inoculation, area of lesions was examined. As a result, the lesion area on the plant treated with the invented compounds (1), (7), (9), (15), (17), (18), (20), (25), (27) and (28) was less than 10% of the lesion area on the untreated plant.

1. An amide compound represented by the formula (I): 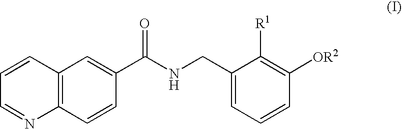
wherein R¹ is a hydrogen atom or a fluorine atom, and R² is a C3-C8 linear alkenyl group or a C3-C8 linear alkynyl group.

2. The amide compound according to claim 1, wherein R¹ is a fluorine atom, and R² is a C5-C7 linear alkenyl group.

3. The amide compound according to claim 1, wherein R¹ is a fluorine atom, and R² is a 4-pentenyl group, 5-hexenyl group or 6-heptenyl group.

4. The amide compound according to claim 1, wherein R¹ is a fluorine atom, and R² is a C5-C7 linear alkynyl group.

5. The amide compound according to claim 1, wherein R¹ is a fluorine atom, and R² is a 4-pentynyl group, 5-hexynyl group or 6-heptynyl group.

6. The amide compound according to claim 1, wherein R¹ is a hydrogen atom, and R² is a C5-C7 linear alkenyl group.

7. The amide compound according to claim 1, wherein R¹ is a hydrogen atom, and R² is a 4-pentenyl group, 5-hexenyl group or 6-heptenyl group.

8. The amide compound according to claim 1, wherein R¹ is a hydrogen atom, and R² is a C5-C7 linear alkynyl group.

9. The amide compound according to claim 1, wherein R¹ is a hydrogen atom, and R² is a 4-pentynyl group, 5-hexynyl group or 6-heptynyl group.

10. A composition for controlling plant diseases, which comprises an amide compound according to claim 1 as an active ingredient.

11. A method for controlling plant diseases, which comprises a step of applying an effective amount of an amide compound according to claim 1 to plants or soil.

12. A method of using an amide compound according to claim 1 for controlling plant diseases comprising applying the amide compound to plants or soil.

13. An amine compound represented by the formula (III), or its salts: 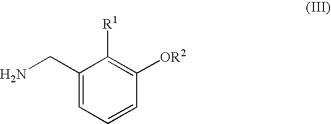
wherein R¹ is a hydrogen atom or a fluorine atom, and R² is a C3-C8 linear alkenyl group or a C3-C8 linear alkynyl group.
